(19) United States
(12) Patent Application Publication
(10) Pub. No.: US 2024/0317693 A1
(43) Pub. Date: Sep. 26, 2024

(54) POLY(TETRAHYDROQUINAZOLINE) AND DERIVED CARBON AEROGELS

(71) Applicant: The Curators of the University of Missouri, Columbia, MO (US)

(72) Inventors: Chariklia Sotiriou-Leventis, Rolla, MO (US); Nicholas Leventis, Worcester, MA (US); Vaibhav Edlabadkar, Rolla, MO (US); Rushi Soni, Rolla, MO (US); Saidulu Gorla, Starkville, MS (US); ABM Shaheen ud Doulah, Rolla, MO (US)

(73) Assignee: The Curators of the University of Missouri, Columbia, MO (US)

(21) Appl. No.: 18/554,062

(22) PCT Filed: Apr. 5, 2022
(86) PCT No.: PCT/US2022/023452

§ 371 Date: Oct. 5, 2023

Related U.S. Application Data
(60) Provisional application No. 63/170,614, filed on Apr. 5, 2021.

(51) Int. Cl. C07D 239/74; C08G 61/12; C08J 3/075

(52) U.S. Cl. CPC C07D 239/74; C08G 61/122; C08J 3/075; C08J 2379/04

(57) ABSTRACT

Novel compounds and polymer aerogels derived from these compounds are provided. The highly porous, monolithic polymer aerogels are extremely robust having high surface areas, large micropore volumes. and high density of nitrogen and oxygen functionalities. Due to these extraordinary properties, the polymer aerogels possess a high carbon dioxide (CO₂) sorption capacity and are highly selective towards CO₂ versus other gases, such as H₂ and N₂. As a result, the polymer aerogels can be used to effectively capture or remove CO₂ from the air and/or from flue gases.

### STATEMENT REGARDING FEDERALLY SPONSORED RESEARCH OR DEVELOPMENT

This invention was made with government support under Contract No. 1530603 awarded by the National Science Foundation and under Contract No. W911NF-14-1-0369 awarded by Army Research Office. The government has certain rights in the invention.

### BACKGROUND

### Related Applications

The present application claims the priority benefit of U.S. Provisional Patent Application Ser. No. 63/170,614, filed Apr. 5, 2021, entitled POLY(TETRAHYDROQUINAZOLINE) AND DERIVED CARBON AEROGELS, incorporated by reference in its entirety herein.

### Field

The present disclosure is concerned with novel compounds and polymer aerogels derived from these compounds and with methods of using the polymer aerogels to capture or remove carbon dioxide (CO₂) from the air and/or from flue gases.

### Description of Related Art

Aerogels are bulk, lightweight, nanostructured, and nanoporous solids. Due to their ultralow densities and high porosity, they exhibit low thermal conductivities and high acoustic impedance and are typically used for thermal and acoustic insulation. Most aerogels, however, are based on silica or metal oxides, and aerogels based on carbon often do not contain a high nitrogen or oxygen content, which can play a role on the properties of porous carbon materials. Particularly, nitrogen-containing functional groups are responsible for adding basic character to porous carbon materials, which tends to improve interaction with gases for gas adsorption applications, such as CO₂ capture or removal. However, nitrogen is typically introduced in porous carbon materials by post-treatment with nitrogen containing molecules, such as ammonia, urea, and melamine nitric acid. The post-treatment method is straightforward, but the derived carbon materials are oftentimes unstable and, in general, show lower surface areas and pore volumes than the untreated carbon materials.

Thus, there is a need for highly porous polymer aerogels comprising carbon and nitrogen that can be used in the carbon capture and storage industry.

### SUMMARY

The present disclosure is broadly concerned with novel compounds and polymer aerogels derived from these compounds. The compound comprises moiety (I):

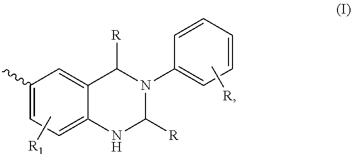

where:
[symbol] represents the point of attachment of the moiety to the rest of the compound;
each R is individually chosen from hydrogen and alkyls; and
each R₁ is individually chosen from hydrogen, halogens, and alkyls.

In another embodiment, a polymer is provided. The polymer comprises recurring units chosen from one or more of

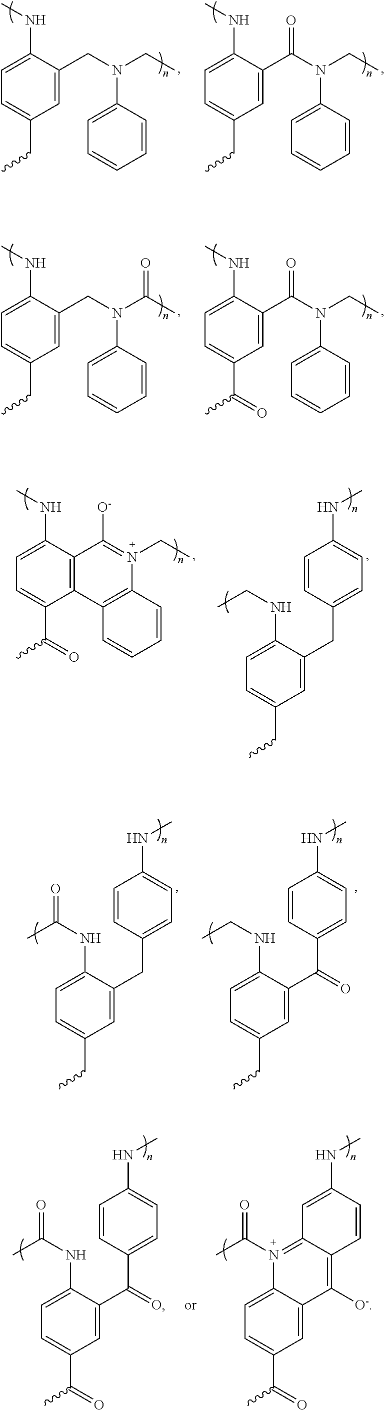

In other embodiment, the polymer comprises recurring units chosen from one or more of

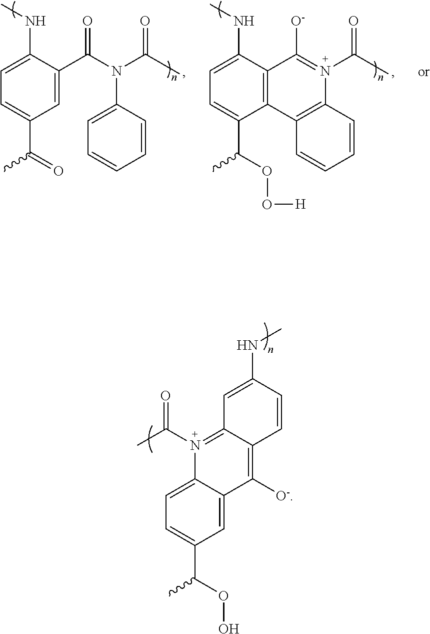

In yet another embodiment, a monomer synthesis process is provided. The monomer synthesis process comprises: (a) reacting anthranilic acid with an aqueous solution comprising an acid and formaldehyde to yield a product comprising 5,5′-methylenebis(2-aminobenzoic acid); (b) reacting said product comprising 5,5′-methylenebis(2-aminobenzoic acid) with a mixture comprising triphosgene and a solvent to yield a product comprising 6,6′-methylenebis(1H-benzo[d][1,3]oxazine-2,4-dione); (c) reacting said product comprising 6,6′-methylenebis(1H-benzo[d][1,3]oxazine-2,4-dione) with a mixture comprising aniline, triethyl orthoformate, and a solvent to yield a product comprising 6,6′-methylenebis(3-phenylquinazolin-4(3H)-one); and (d) reacting said product comprising 6,6′-methylenebis(3-phenylquinazolin-4(3H)-one) with a mixture comprising aluminum chloride, lithium aluminum hydride, and a solvent to yield a product comprising bis(3-phenyl-1,2,3,4-tetrahydroquinazolin-6-yl)methane.

Finally, in yet another embodiment, a polymer aerogel is provided. The polymer aerogel comprises at least about 84.5% by weight carbon; and at least about 3% by weight nitrogen, said % by weight being based on the total weight of the polymer aerogel taken as 100% by weight, said polymer aerogel having a CO₂ sorption capacity of at least about 7 mmol/g.

### BRIEF DESCRIPTION OF THE DRAWINGS

FIG. 1 is a graph showing the modulated differential scanning calorimetry plot for PTHQ-100;

FIG. 2 shows the liquid-state ¹³C attached proton test nuclear magnetic resonance (NMR) spectrum for THQ and the solid-state cross-polarization magic angle spinning ¹³C NMR spectra for THQ, PTHQ-100, PTHQ-240, PTHQ-300, PTHQ-400, and PTHQ-600;

FIG. 3A shows the solid-state ¹⁵N NMR spectra of ¹⁵N-enriched aniline & anthranilic acid THQ, PTHQ-100, and PTHQ-240;

FIG. 3B shows the solid-state ¹⁵N NMR spectra of ¹⁵N-enriched aniline THQ, PTHQ-100, and PTHQ-240;

FIG. 3C shows the solid-state ¹⁵N NMR spectra of ¹⁵N-enriched anthranilic acid THQ, PTHQ-100, and PTHQ-240;

FIG. 4 shows the ¹H-¹⁵N heteronuclear single quantum coherence NMR spectrum of THQ;

FIG. 5A shows the solid-state ¹⁵N NMR spectra of THQ with a contact time of 3,000 μs;

FIG. 5B shows the solid-state ¹⁵N NMR spectra of THQ with a contact time of 5 μs;

FIG. 6A is a schematic depiction of a proposed mechanism for HCl-catalyzed ring opening polymerization of THQ;

FIG. 6B is a schematic depiction of another proposed mechanism for HCl-catalyzed ring opening polymerization of THQ;

FIG. 7 is a schematic depiction of a proposed mechanism for oxidative aromatization of PTHQ-100;

FIG. 8 shows the solid-state ¹⁵N NMR spectrum of a control experiment used to calculate the amide:imide response factor for ¹⁵N-enriched aniline PTHQ-240 and ¹⁵N-enriched anthranilic acid PTHQ-240;

FIG. 9 shows the Fourier-transform infrared spectra for THQ, PTHQ-100, PTHQ-240, PTHQ-300, PTHQ-400, and PTHQ-600;

FIG. 10A shows the X-ray photoelectron spectroscopy (XPS) spectra of N 1 s (left) and O 1s (right) peaks for C-PTHQ-800;

FIG. 10B shows the XPS spectra of N 1 s (left) and O 1 s (right) peaks for EC-PTHQ-1000;

FIG. 11 is a photograph with an aerial view of a PTHQ-100 aerogel monolith, a PTHQ-240 aerogel monolith, a C-PTHQ-800 aerogel monolith, an EC-PTHQ-1000 aerogel monolith, and a PTHQ-100 aerogel monolith processed at 800° C.;

FIG. 12 is a graph showing the representative N₂-sorption isotherms at 77 K of the PTHQ-100 and PTHQ-240 aerogels;

FIG. 13 is a graph showing the pore size distribution data calculated using the Barrett, Joyner, and Halenda (BJH) method for the PTHQ-100 and PTHQ-240 aerogels;

FIG. 14 is a series of scanning electron microscope photographs of the PTHQ-100, PTHQ-240, C-PTHQ-800, and EC-PTHQ-1000 aerogels;

FIG. 15 is a graph showing the representative low-and medium-pressure N₂-sorption isotherms at 77 K of the C-PTHQ-800 and EC-PTHQ-1000 aerogels;

FIG. 16A is a graph showing the representative CO₂ adsorption isotherms at 273 K of the C-PTHQ-800 and EC-PTHQ-1000 aerogels;

FIG. 16B is a graph showing the representative CO₂ adsorption isotherms at 298 K of the C-PTHQ-800 and EC-PTHQ-1000 aerogels;

FIG. 16C is a comparative graph showing the CO₂ adsorption by the C-PTHQ-800 and EC-PTHQ-1000 aerogels versus other sorbents at 273 K;

FIG. 17A is a graph showing the micropore size distributions <1 nm calculated using the density-functional theory method (assuming slit pores) as applied to CO₂ adsorption data for the C-PTHQ-800 and EC-PTHQ-1000 aerogels;

FIG. 17B is a graph showing the mesopore size distributions calculated using the BJH method as applied to N₂ sorption data for the C-PTHQ-800 and EC-PTHQ-1000 aerogels;

FIG. 18 is a graph showing the isosteric heats of CO₂ adsorption for the C-PTHQ-800 and EC-PTHQ-1000 aerogels;

FIG. 19A is a line graph showing the selectivity of the C-PTHQ-800 aerogel toward CO₂ adsorption versus CH₄, N₂, and H₂;

FIG. 19B is a line graph showing the selectivity of the EC-PTHQ-1000 aerogel toward CO₂ adsorption versus CH₄, N₂, and H₂; and

FIG. 19C is a bar graph showing the selectivity of the C-PTHQ-800 and EC-PTHQ-1000 aerogels toward CO₂ adsorption versus CH₄, N₂, and H₂.

### DETAILED DESCRIPTION

The present disclosure is concerned with novel compounds and polymer aerogels derived from these compounds and with methods of using the polymer aerogels to capture and/or remove carbon dioxide (CO₂) from the air and/or from flue gases.

### Novel Compounds

The novel compound of the present disclosure comprises the moiety (I):

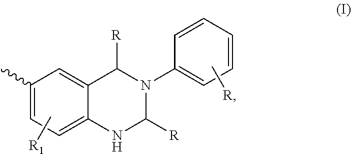

where:
[symbol] represents the point of attachment of the moiety to the rest of the compound;
each R is individually chosen (i.e., each R can be the same or different) from hydrogen and alkyls (preferably C₁-C₁₂, more preferably C₁-C₆, and even more preferably C₁-C₃); and
each R₁ is individually chosen (i.e., each R₁ can be the same or different) from hydrogen, halogens (preferably —Cl, —F, —Br, and/or —I), and alkyls (preferably C₁-C₁₂, more preferably C₁-C₆, and even more preferably C₁-C₃).

Preferably, at least one R group in moiety (I) is hydrogen, and in more preferred embodiments, each R group is hydrogen. In other embodiments, at least one R₁ group in moiety (I) is hydrogen, and preferably each R₁ group in moiety (I) is hydrogen. In another embodiment, at least one R group is a hydrogen and at least on R₁ group is a hydrogen. In one preferred embodiment, each R group and R₁ group in moiety (I) is hydrogen.

Bearing the above-described moiety (I), the compound, in some embodiments, may comprise at least about 2.5%, preferably at least about 6%, by weight nitrogen, based upon the total weight of the compound taken as 100% by weight. Furthermore, the compound comprises about 6% to about 18% by weight nitrogen, preferably about 8% to about 16% by weight nitrogen, and more preferably about 11.5% to about 14.5% by weight nitrogen, based upon the total weight of the compound taken as 100% by weight.

In one embodiment, the compound is essentially free of oxygen. In other words, the compound comprises less than about 6.5% by weight, preferably less than about 3% by weight, more preferably less than about 1% by weight, and even more preferably about 0% by weight of oxygen, wherein the weight % percentages are based upon the total weight of the compound taken as 100% by weight. In a preferred embodiment, the compound comprises the structure:

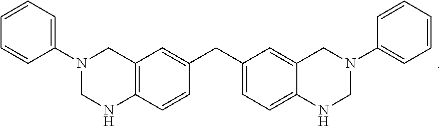 [SMILES: c1ccc(N2CNc3ccc(Cc4ccc5c(c4)CN(c4ccccc4)CN5)cc3C2)cc1]

### Compound Synthesis

The novel compounds can be synthesized using a process that, in at least one embodiment, starts from commercially available anthranilic acid. When anthranilic acid is used as a starting point, the synthesis process comprises reacting anthranilic acid with formaldehyde to form 5,5′-methylenebis(2-aminobenzoic acid) (also referred to as 4,4′-methylene bisanthranilic acid) through an acid-catalyzed reaction. Preferably, anthranilic acid is added to an aqueous solution comprising an acid, such as concentrated hydrochloric acid, and formaldehyde. In more preferred embodiments, anthranilic acid is initially added to an aqueous solution comprising an acid, and the mixture is heated with stirring to a temperature of from about 40° C. to about 60° C. for a time period of from about 5 minutes to about 20 minutes. Then, formaldehyde is added to the mixture. The molar ratio of anthranilic acid to formaldehyde is preferably about 1:1 to about 1:1.8, and more preferably about 1:1.6 to about 1:0.57.

Regardless of how anthranilic acid or formaldehyde are added, the solution is then heated with stirring preferably to a temperature of from about 50° C. to about 90° C., and more preferably about 60° C. to about 80° C., for a time period of from about 1 hour to about 7 hours, and more preferably about 3 hours to about 5 hours. The reaction solution is cooled to room temperature, and 5,5′-methylenebis(2-aminobenzoic acid) is precipitated from the solution, preferably by neutralizing the solution with a concentrated aqueous base to a pH of about 6.5 to about 7.5. The precipitated 5,5′-methylenebis(2-aminobenzoic acid) is filtered and washed, preferably with water. The obtained 5,5′-methylenebis(2-aminobenzoic acid) can be air-dried or dried in a vacuum for a time period of from about 10 hours to about 24 hours.

In a next step, the synthesis process further comprises reacting the obtained 5,5′-methylenebis(2-aminobenzoic acid) with triphosgene to yield 6,6′-methylenebis(1H-benzo[d][1,3]oxazine-2,4-dione) (also referred to as bisamide-anhydride). In one embodiment, commercially available 5,5′-methylenebis(2-aminobenzoic acid) can be utilized. The molar ratio of 5,5′-methylenebis(2-aminobenzoic acid) to triphosgene is preferably about 1:0.72 to about 1:0.70, and more preferably about 1:0.68 to about 1:0.66. In preferred embodiments, 5,5′-methylenebis(2-aminobenzoic acid) and triphosgene are reacted in a solvent, preferably dioxane. The mixture is then heated to reflux at a temperature of from about 80° C. to about 120° C., and preferably about 90° C. to about 110° C., for a time period of from about 4 hours to about 8 hours, and preferably about 5 hours to about 7 hours. The precipitated 6,6′-methylenebis(1H-benzo[d][1,3]oxazine-2,4-dione) is filtered and washed, preferably with dioxane. The obtained 6,6′-methylenebis(1H-benzo[d][1,3]oxazine-2,4-dione) can be air-dried or dried in a vacuum, preferably at a temperature of from about 70° C. to about 90° C. for a time period of from about 6 hours to about 24 hours.

In a third step, the synthesis process further comprises reacting the obtained 6,6′-methylenebis(1H-benzo[d][1,3]oxazine-2,4-dione) with aniline and triethyl orthoformate to yield 6,6′-methylenebis(3-phenylquinazolin-4(3H)-one). In one embodiment, commercially available 6,6′-methylenebis(1H-benzo[d][1,3]oxazine-2,4-dione) can be utilized. The molar ratio of 6,6′-methylenebis(1H-benzo[d][1,3]oxazine-2,4-dione) to aniline is preferably about 1:3.3 to about 1:3.2, and more preferably about 1:3.1 to about 1:3. Furthermore, the molar ratio of 6,6′-methylenebis(1H-benzo[d][1,3]oxazine-2,4-dione) to triethyl orthoformate is preferably about 1:3.3 to about 1:3.2, and more preferably about 1:3.1 to about 1:3.

In preferred embodiments, 6,6′-methylenebis(1H-benzo[d][1,3 ]oxazine-2,4-dione), aniline, and triethyl orthoformate are reacted in a solvent, examples of which are chosen from ethanol, methanol, isopropanol, and mixtures thereof, with ethanol being preferred. The mixture is then heated with stirring to a temperature of from about 60° C. to about 100° C., and preferably about 70° C. to about 90° C., for a time period of from about 14 hours to about 22 hours, and preferably about 16 hours to about 20 hours. The reaction mixture is cooled to room temperature, and the precipitated 6,6′-methylenebis(3-phenylquinazolin-4(3H)-one) is filtered and washed, preferably with ethanol. The obtained 6,6′-methylenebis(3-phenylquinazolin-4(3H)-one) can be air-dried or dried in a vacuum, preferably at a temperature of from about 70° C. to about 90° C. for a time period of from about 6 hours to about 24 hours.

In a fourth step, the synthesis process further comprises reacting the obtained 6,6′-methylenebis(3-phenylquinazolin-4(3H)-one) with one or more reducing agents, such as aluminum chloride and/or lithium aluminum hydride, to yield bis(3-phenyl-1,2,3,4-tetrahydroquinazolin-6-yl)methane (THQ) through a reduction reaction. In one embodiment, commercially available 6,6′-methylenebis(3-phenylquinazolin-4(3H)-one) can be utilized. Preferably, 6,6′-methylenebis(3-phenylquinazolin-4(3H)-one) is introduced to a solution and/or suspension comprising aluminum chloride, lithium aluminum hydride, and a solvent, such as tetrahydrofuran (THF). More preferably, the solution and/or suspension is first stirred at a temperature of from about −5° C. to about 5° C. for a time period of from about 30 minutes to about 2 hours before 6,6′-methylenebis(3-phenylquinazolin-4(3H)-one) is introduced to the solution and/or suspension. The molar ratio of 6,6′-methylenebis(3-phenylquinazolin-4(3H)-one) to aluminum chloride is preferably about 1:2.6 to about 1:2.4, and more preferably about 1:2.2 to about 1:2. Furthermore, the molar ratio of 6,6′-methylenebis(3-phenylquinazolin-4(3H)-one) to lithium aluminum hydride is preferably about 1:3.6 to about 1:3.4, and more preferably about 1:3.2 to about 1:3.

Regardless of how 6,6′-methylenebis(3-phenylquinazolin-4(3H)-one) is introduced, the mixture is stirred at a temperature of from about −5° C. to about 5° C. for a time period of from about 1 hour to about 3 hours, and the produced solids are filtered and washed, preferably with THF. The filtrate and washing liquid are then preferably combined to form a second mixture, and the solvent is removed from the second mixture using an evaporator at a temperature of from about 35° C. to about 45° C. and under a pressure of about 5 KPa to about 6 KPa for a time period of from about 30 minutes to about 45 minutes, yielding a crude product. The crude product is then preferably heated at a temperature of from about 65° C. to about 70° C. for a time period of from about 1 hour to about 3 hours in a solvent, preferably methanol. The THQ precipitate is filtered and washed, preferably with methanol. The obtained THQ can be air-dried or dried in a vacuum, preferably at a temperature of from about 70° C. to about 90° C. for a time period of from about 6 hours to about 24 hours. Those skilled in the art will understand how to modify the foregoing synthesis process to form other compounds comprising moiety (I).

In most preferred embodiments, the novel compounds are synthesized using a process as shown in Scheme 1.

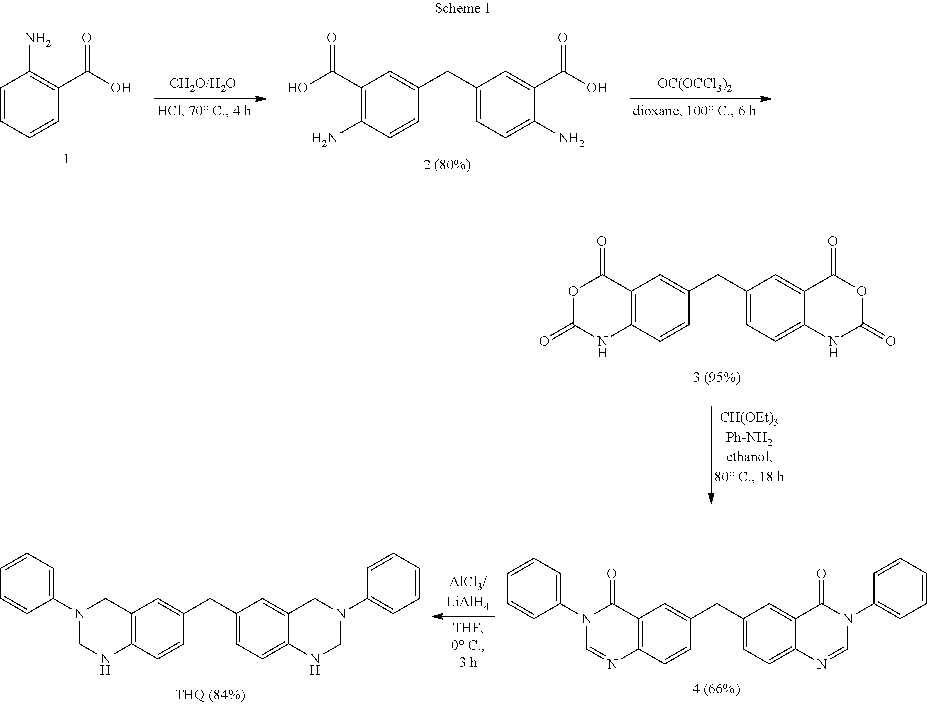

### Polymer Wet Gels and Aerogels

The polymers of the present disclosure are preferably derived from a compound comprising moiety (I), and more preferably derived from THQ. Regardless of its derivative compound, the polymer preferably comprises recurring units chosen from one or more of:

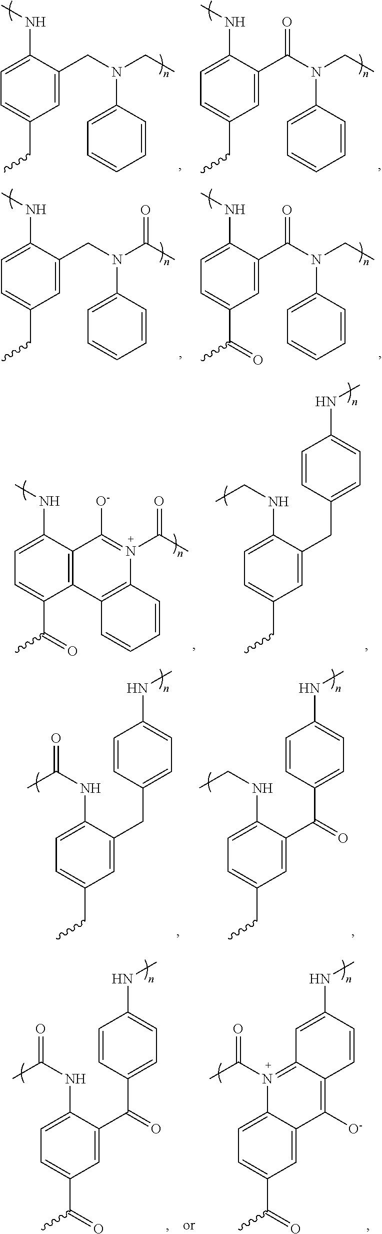

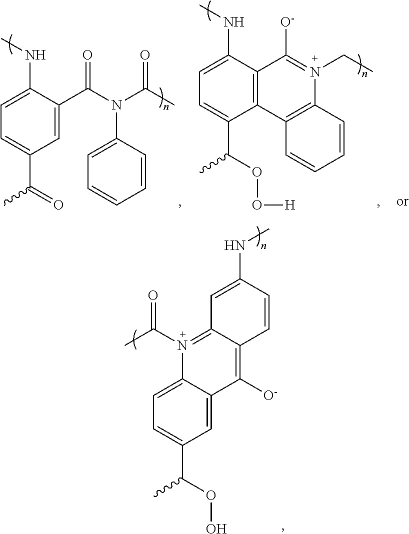

where n is 1 to about 20, preferably 1 to about 15, and more preferably 1 to about 10.

### 1. Partially Oxidized Polymer Wet Gels and Aerogels

In some preferred embodiments, the polymer is referred to as a partially oxidized polymer because it comprises about 2% to about 8% by weight oxygen, preferably about 3% to about 7% by weight oxygen, and more preferably about 4% to about 6% by weight oxygen, based upon the total weight of the polymer taken as 100% by weight. The partially oxidized polymer also comprises about 8% to about 16% by weight nitrogen, preferably about 9.5% to about 14.5% by weight nitrogen, and more preferably about 11% to about 12% by weight nitrogen, based upon the total weight of the polymer taken as 100% by weight.

Preferably, the partially oxidized polymers comprise recurring units chosen from one or more of:

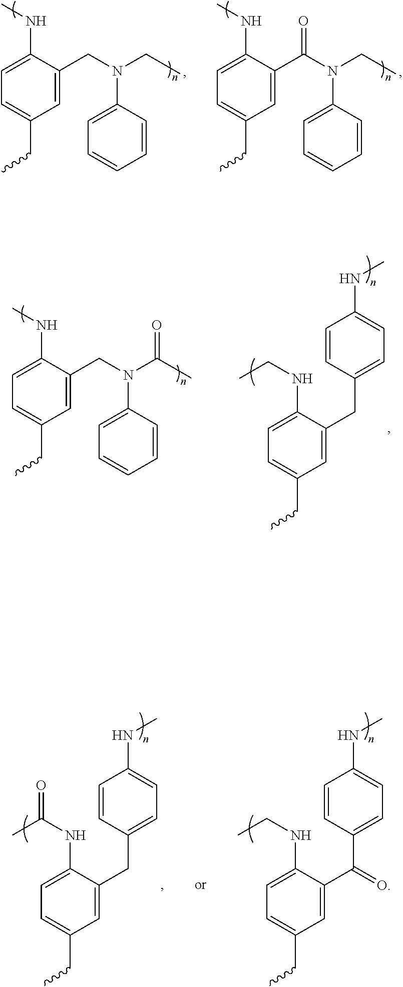

In more preferred embodiments, partially oxidized polymers comprise recurring units chosen from one or more of:

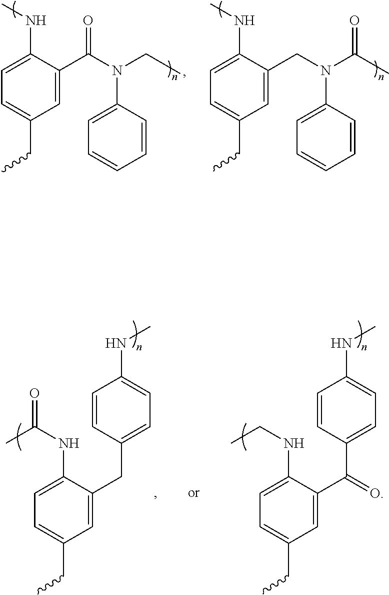

It will be appreciated that, in embodiments where THQ is used, the partially oxidized polymer can be polymerized through at least two different ring-opening polymerization reaction mechanisms. In one embodiment, the mechanism of polymerization for the partially oxidized polymers is as shown in Scheme 2 (“Mechanism A”).

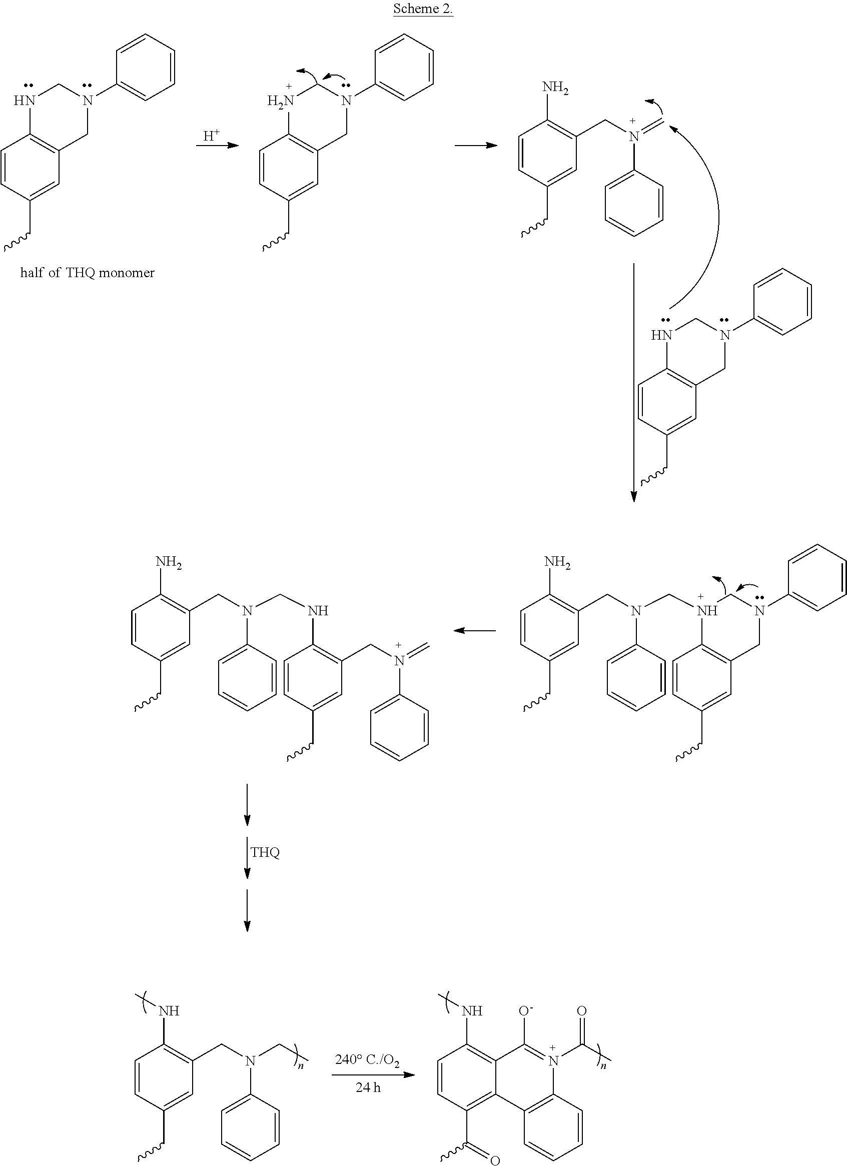

Partially oxidized polymers produced by Mechanism A comprise recurring units chosen from one or more of:

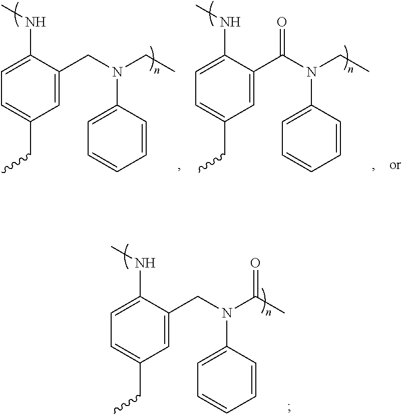

and more preferably

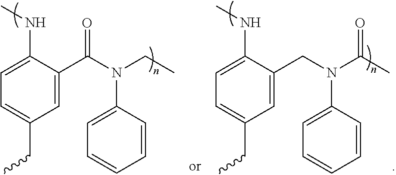

In another embodiment, the mechanism of polymerization for the partially oxidized polymers is as shown in Scheme 3 (“Mechanism B”).

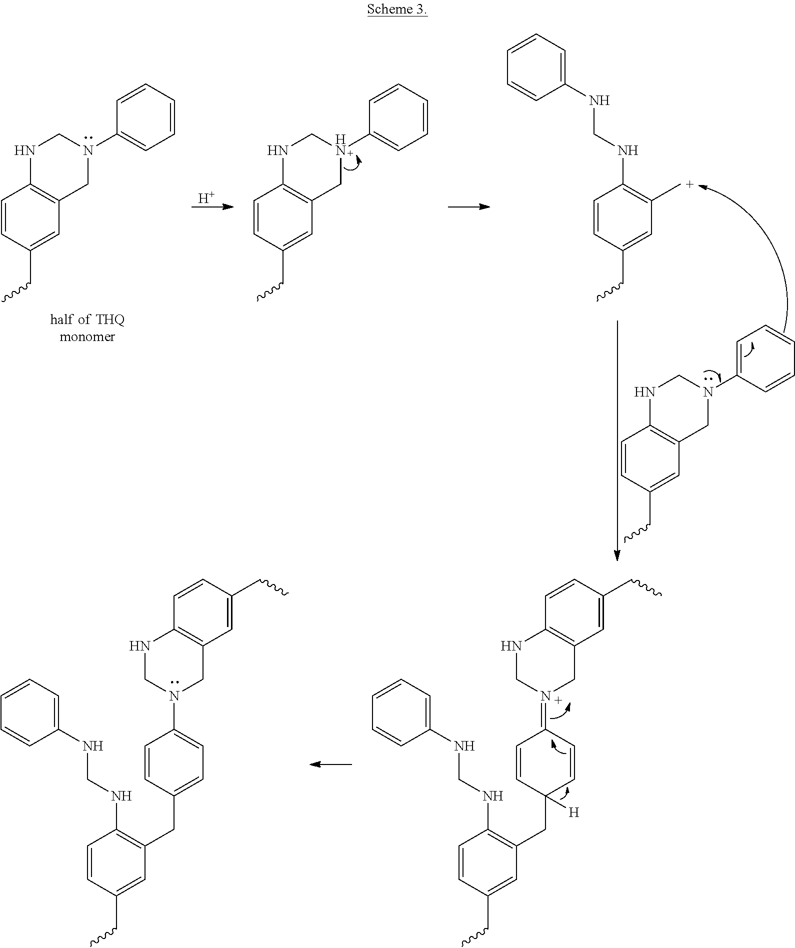

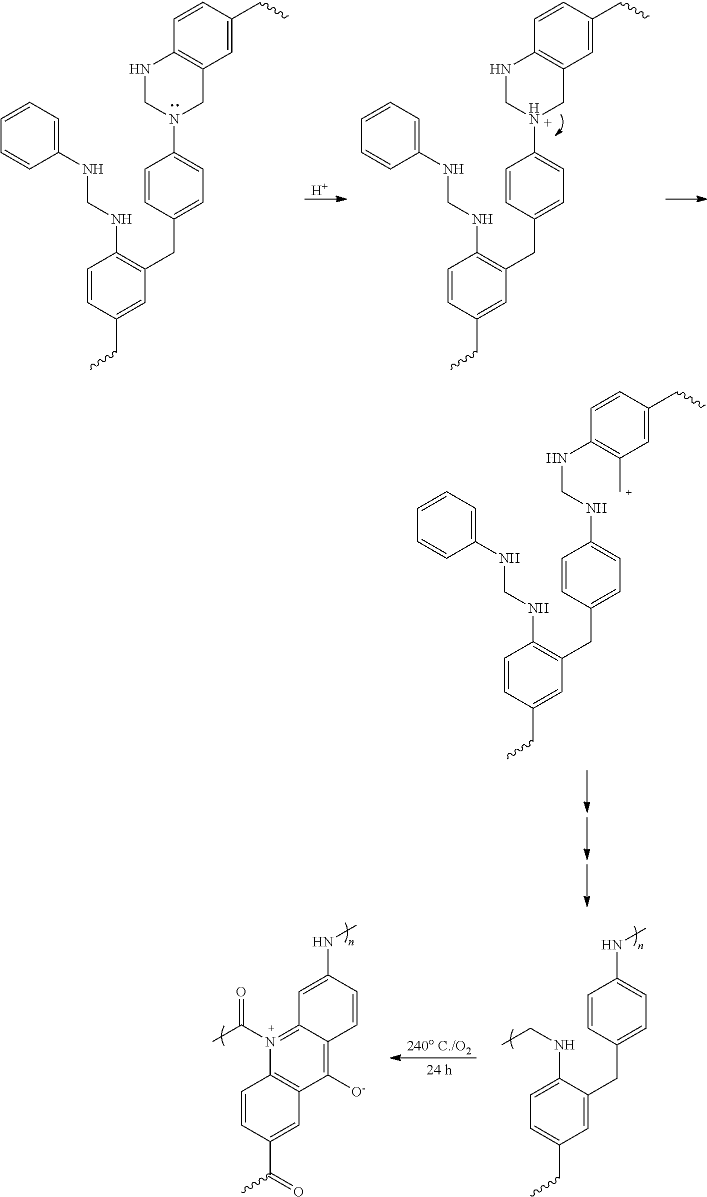

Partially oxidized polymers produced by Mechanism B comprise recurring units chosen from one or more of:

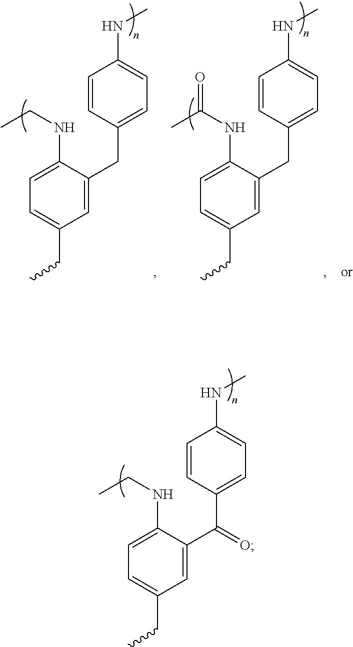

and more preferably

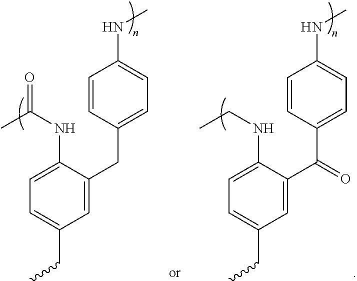

The partially oxidized polymers may be synthesized as a colloid, such as a wet gel, solid, liquid, solution, preferably as a colloid, and more preferably as a wet gel. To synthesize a partially oxidized polymer wet gel, a compound comprising moiety (I), more preferably THQ, is polymerized through a ring-opening polymerization reaction, preferably through an acid-catalyzed ring-opening polymerization reaction. Preferably, THQ is introduced to a solution comprising an acid, such as concentrated hydrochloric acid, and a gelation solvent, examples of which are chosen from dimethylformamide (DMF), dimethylsulfoxide (DMSO), dimethylacetamide (DMAc), preferably DMF. The weight percent concentration of THQ in the solution is about 10% w/w to about 30% w/w, preferably about 15% w/w to about 25% w/w. The resulting solution is stirred for a time period of from about 5 minutes to about 15 minutes at room temperature and added to a glass tube. The tube is purged with a purge gas, such as nitrogen, CO₂, argon, and helium, preferably argon, for a time period of from about 30 seconds to about 2 minutes. Subsequently, the tube is degassed using a freeze-pump-thaw cycle at a temperature of from about 74 K to about 80 K, and the freeze-pump-thaw cycle is repeated one to three times, preferably two times. At the end of the last freeze-pump-thaw cycle, the tube is flame-sealed, preferably under vacuum, and the resulting solution (inside the sealed tube) is left for gelation at a temperature of from about 70° C. to about 130° C., preferably from about 85° C. to about 115° C., more preferably from about 95° C. to about 105° C., and even more preferably about 100° C. for a time period of from about 24 hours to about 120 hours, preferably from about 48 hours to about 96 hours, more preferably from about 60 hours to about 84 hours, and even more preferably about 72 hours.

Then, the partially oxidized polymer wet gel is retrieved from the tube and washed with one or more solvents, preferably about 5 to about 9 times for a time period of from about 3 hours to about 5 hours each time with DMF and about 4 times to about 8 times for a time period of from about 3 hours to about 5 hours each time with acetone. The amount of solvent for each wash is three times to five times the volume of the partially oxidized polymer wet gel.

Preferably, the partially oxidized polymer wet gels can be made into partially oxidized polymer aerogels, which comprise, consist essentially of, or even consist of a partially oxidized polymer. Those skilled in the art will readily understand that partially oxidized polymer cryogels and/or xerogels can be made from partially oxidized polymer wet gels according to methods known in the art.

To synthesize the partially oxidized polymer aerogel, a partially oxidized polymer wet gel is dried using a supercritical drying process or a subcritical drying process, preferably a supercritical drying process. When a supercritical drying process is used, the gelation solvent of the partially oxidized polymer wet gel is replaced with a supercritical fluid, preferably supercritical CO₂. The supercritical fluid is then vented off as a gas.

### 2. Fully Oxidized Polymer Aerogels

In other preferred embodiments, the polymer is referred to as a fully oxidized polymer because it comprises about 10% to about 24.5% by weight oxygen, preferably about 14% to about 22% by weight oxygen, and more preferably about 16% to about 20% by weight oxygen, based upon the total weight of the polymer taken as 100% by weight. The fully oxidized polymer comprises about 8% to about 16% by weight nitrogen, preferably about 9.5% to about 14.5% by weight nitrogen, and more preferably about 11% to about 12% by weight nitrogen, based upon the total weight of the polymer taken as 100% by weight.

Preferably, fully oxidized polymers comprise recurring units chosen from one or more of:

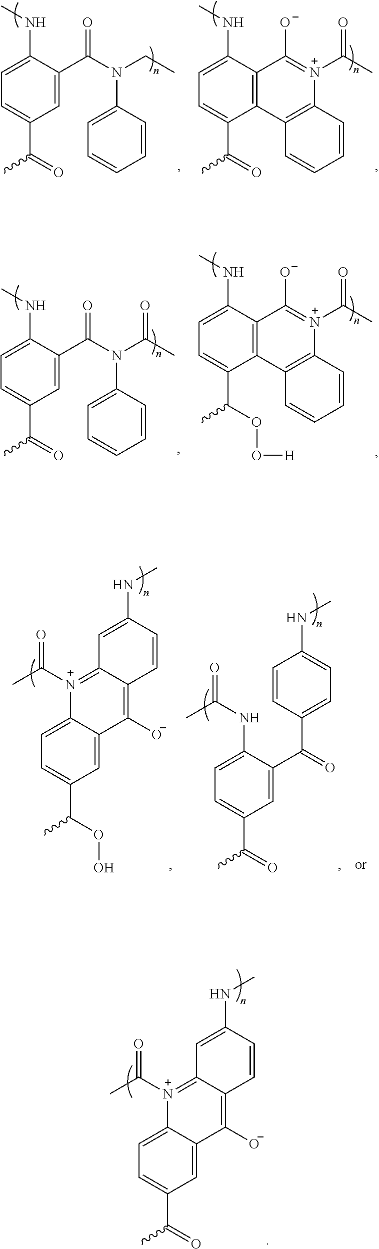

In preferred embodiments, where the fully oxidized polymer is synthesized from a partially oxidized polymer produced by Mechanism A, the fully oxidized polymer comprises recurring units chosen from one or more of:

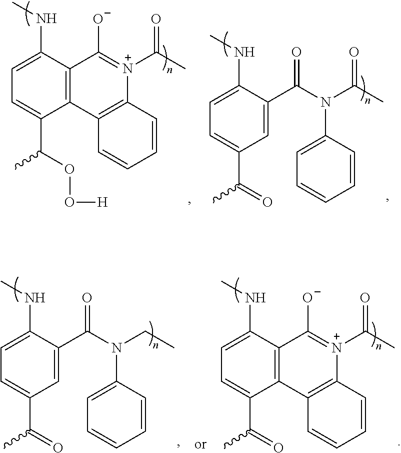

In other preferred embodiments, where the fully oxidized polymer is synthesized from a partially oxidized polymer produced by Mechanism B, the fully oxidized polymer comprises recurring units chosen from one or more of:

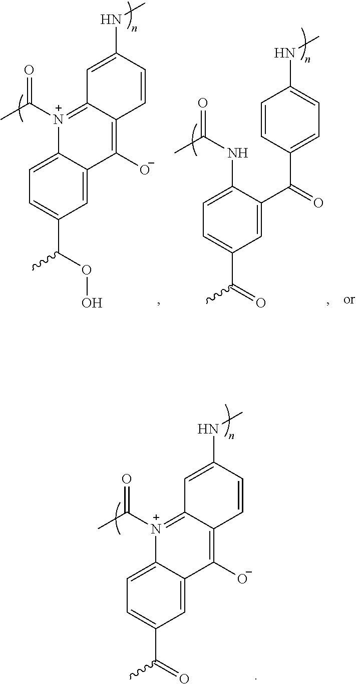

The fully oxidized polymers may be synthesized as a colloid, solid, such as an aerogel, liquid, solution, preferably as a solid, and more preferably as an aerogel. In embodiments where the fully oxidized polymers are synthesized as aerogels, the fully oxidized polymer aerogels comprise, consist essentially of, or even consist of fully oxidized polymers. To synthesize the fully oxidized polymer aerogel, a partially oxidized polymer wet gel or aerogel, preferably a partially oxidized polymer wet gel or aerogel produced by Mechanism A or Mechanism B, is oxidized through a ring-fusion aromatization reaction. The partially oxidized polymer wet gel or aerogel is subjected to heat treatment in air or under oxygen, preferably oxygen, at a flow rate of about 0.05 L min to about 1 L min⁻¹, preferably about 0.09 L min⁻¹ to about 0.7 L min⁻¹. The polymer wet gel or aerogel is heated to a temperature of from about 200° C. to about 280° C., preferably from about 215° C. to about 265° C., more preferably from about 230° C. to about 250° C., and even more preferably about 240° C. at a heating rate of from about 1° C. min⁻¹ to about 4° C. min⁻¹. Regardless of the temperature used, the polymer wet gel or aerogel is heated for a time period of from about 12 hours to about 36 hours, preferably from about 18 hours to about 30 hours, more preferably from about 22 hours to about 26 hours, and even more preferably about 24 hours.

In some most preferred embodiments, where the fully oxidized polymer is synthesized from a partially oxidized polymer produced by Mechanism A, the ring-fusion aromatization reaction proceeds as shown in Scheme 4A.

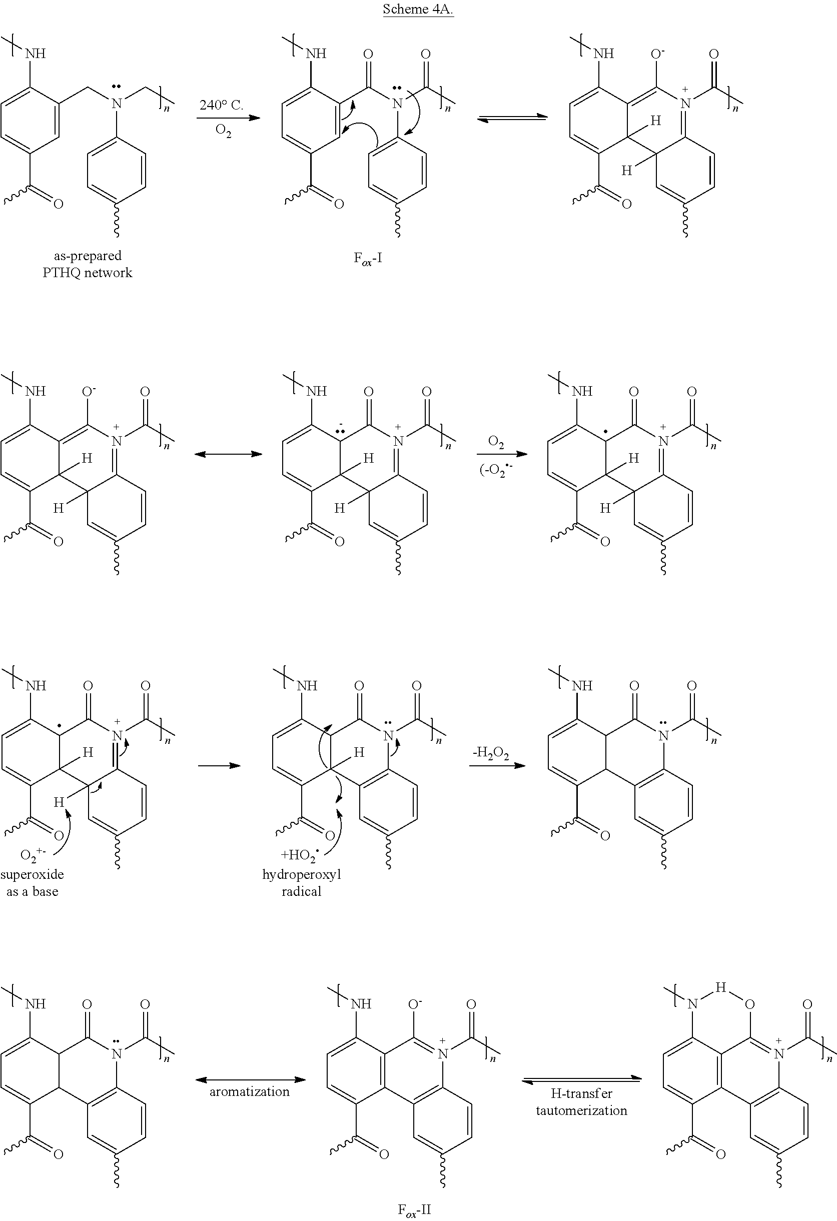

In other most preferred embodiments, where the fully oxidized polymer is synthesized from a partially oxidized polymer produced by Mechanism B, the ring-fusion aromatization reaction proceeds as shown in Scheme 4B.

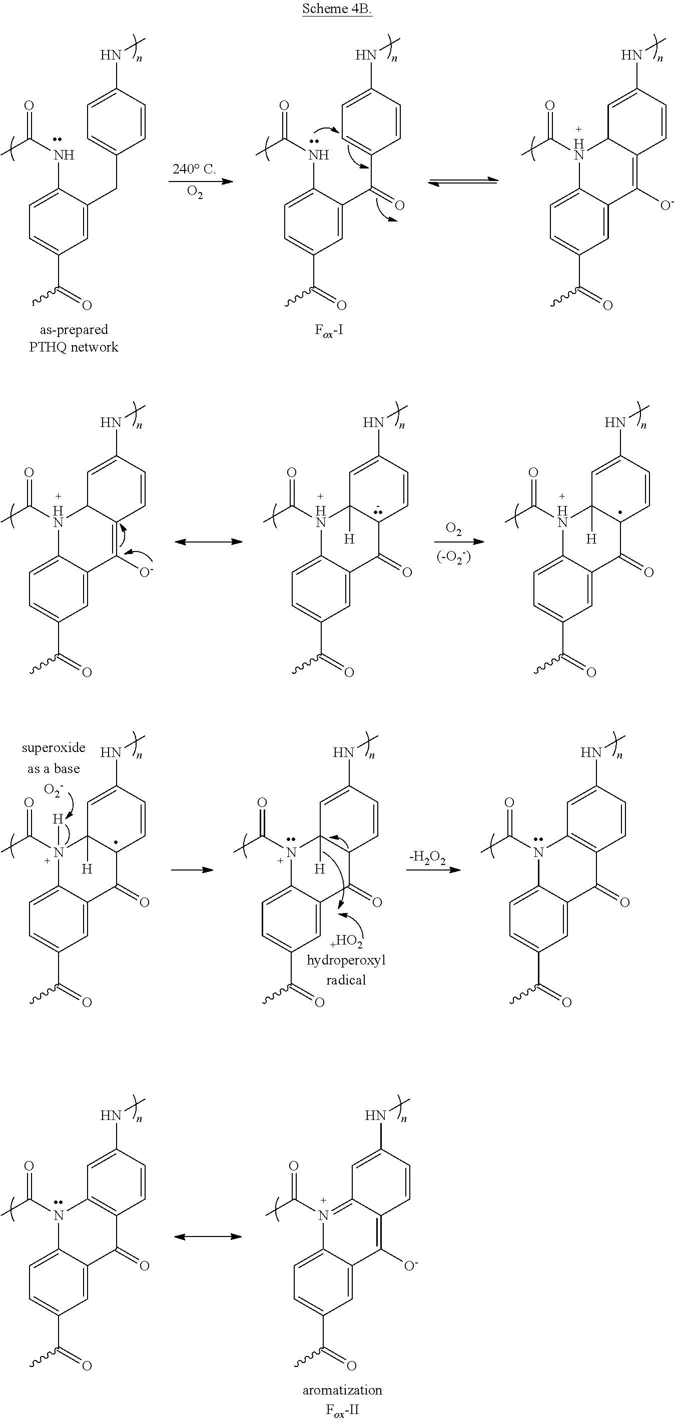

### 3. Carbon Polymer Aerogels

The carbon polymer aerogels are derived from partially oxidized polymer aerogels or fully oxidized polymer aerogels, preferably from fully oxidized polymer aerogels. It will be appreciated that, when carbon polymer aerogels derived from fully oxidized polymer aerogels are processed at certain temperatures, the carbon polymer aerogels notably possess different chemical compositions. In one embodiment, where the carbon polymer aerogel is processed below 500° C., the carbon polymer aerogel comprises about 63% to about 76% by weight carbon, preferably about 65% to about 74% by weight carbon, and more preferably about 67% to about 72% by weight carbon, based upon the total weight of the aerogel taken as 100% by weight. Furthermore, in this embodiment, the carbon polymer aerogel comprises about 2% to about 5% by weight hydrogen, preferably about 2.5% to about 4.5% by weight hydrogen, and more preferably about 3% to about 4% by weight hydrogen, based upon the total weight of the aerogel taken as 100% by weight. Additionally, in this embodiment, the carbon polymer aerogel comprises about 6% to about 15% by weight nitrogen, preferably about 8.5% to about 12.5% by weight nitrogen, and more preferably about 9.5% to about 11.5% by weight nitrogen, based upon the total weight of the aerogel taken as 100% by weight. In this embodiment, the carbon polymer aerogel comprises about 10% to about 23% by weight oxygen, preferably about 12% to about 21% by weight oxygen, and more preferably about 14% to about 19% by weight oxygen, based upon the total weight of the aerogel taken as 100% by weight.

In another embodiment, where the carbon polymer aerogel is processed from 500° C. to 700° C., the carbon polymer aerogel comprises about 73% to about 87% by weight carbon, preferably about 75% to about 84% by weight carbon, and more preferably about 77% to about 82% by weight carbon, based upon the total weight of the aerogel taken as 100% by weight. Furthermore, in this embodiment, the carbon polymer aerogel comprises about 0.5% to about 4% by weight hydrogen, preferably about 1% to about 3.5% by weight hydrogen, and more preferably about 1.5% to about 3% by weight hydrogen, based upon the total weight of the aerogel taken as 100% by weight. Additionally, in this embodiment, the carbon polymer aerogel comprises about 5% to about 15% by weight nitrogen, preferably about 7% to about 13% by weight nitrogen, and more preferably about 9% to about 11% by weight nitrogen, based upon the total weight of the aerogel taken as 100% by weight. In this embodiment, the carbon polymer aerogel comprises about 4% to about 12% by weight oxygen, preferably about 6% to about 10% by weight oxygen, and more preferably about 7% to about 9% by weight oxygen, based upon the total weight of the aerogel taken as 100% by weight.

In preferred embodiments, where the carbon polymer aerogel is processed above 700° C., the carbon polymer aerogel comprises at least about 83.5%, preferably at least about 84%, by weight carbon, based upon the total weight of the aerogel taken as 100% by weight. In other preferred embodiments, where the carbon polymer aerogel is processed above 700° C., the carbon polymer aerogel comprises at least about 5.5%, preferably at least about 6%, by weight nitrogen, based upon the total weight of the aerogel taken as 100% by weight. In these embodiments, the carbon polymer aerogel comprises about 78% to about 90% by weight carbon, preferably about 80% to about 88% by weight carbon, and more preferably about 82% to about 86% by weight carbon, based upon the total weight of the aerogel taken as 100% by weight. Furthermore, in these embodiments, the carbon polymer aerogel comprises about 0.05% to about 2.5% by weight hydrogen, preferably about 0.1% to about 2% by weight hydrogen, and more preferably about 0.5% to about 1.5% by weight hydrogen, based upon the total weight of the aerogel taken as 100% by weight. Additionally, in these embodiments, the carbon polymer aerogel comprises about 3% to about 11% by weight nitrogen, preferably about 5% to about 9% by weight nitrogen, and more preferably about 6% to about 8% by weight nitrogen, based upon the total weight of the aerogel taken as 100% by weight. In these embodiments, the carbon polymer aerogel comprises about 5% to about 12% by weight oxygen, preferably about 7% to about 10% by weight oxygen, and more preferably about 6% to about 9% by weight oxygen, based upon the total weight of the aerogel taken as 100% by weight.

To synthesize the carbon polymer aerogels, a partially oxidized polymer aerogel or a fully oxidized polymer aerogel, preferably a fully oxidized polymer aerogel, is carbonized by subjecting the aerogel to heat treatment under nitrogen or an inert gas, such as argon, at a flow rate of from about 0.05 L min⁻¹ to about 1 L min⁻¹, preferably about 0.09 L min⁻¹ to about 0.7 L min⁻¹. The partially oxidized polymer aerogel or fully oxidized polymer aerogel is heated to a temperature of from about 250° C. to about 950° C. at a heating rate of from about 1° C. min⁻¹ to about 4° C. min⁻¹. Specifically, in one embodiment, the partially oxidized polymer aerogel or fully oxidized polymer aerogel is heated to a temperature of from about 250° C. to about 500° C., preferably about 300° C. to about 400° C., forming a carbon polymer aerogel processed below 500° C. In another embodiment, the partially oxidized polymer aerogel or fully oxidized polymer aerogel is heated to a temperature of from about 500° C. to about 700° C., preferably about 550° C. to about 650° C., forming a carbon polymer aerogel processed between 500° C. and 700° C. In preferred embodiments, the partially oxidized polymer aerogel or fully oxidized polymer aerogel is heated to a temperature of from 700° C. to about 950° C., preferably from about 700° C. to about 900° C., more preferably from about 750° C. to about 850° C., and even more preferably about 800° C., forming a carbon polymer aerogel processed above 700° C.

Regardless of what temperature is used, the partially oxidized polymer aerogel or fully oxidized polymer aerogel is heated for a time period of from about 2 hours to about 8 hours, preferably from about 3 hours to about 7 hours, more preferably from about 4 hours to about 6 hours, and even more preferably about 5 hours.

### 4. Etched-Carbon Polymer Aerogels

The etched-carbon polymer aerogels are derived from fully oxidized polymer aerogels or carbon polymer aerogels, preferably carbon polymer aerogels, and more preferably carbon polymer aerogels processed above 700° C. In preferred embodiments, the etched-carbon polymer aerogel comprises at least about 84.5%, preferably at least about 85%, by weight carbon, based upon the total weight of the aerogel taken as 100% by weight. In other preferred embodiments, the etched-carbon polymer aerogel comprises at least about 3%, preferably at least about 3.5%, by weight nitrogen, based upon the total weight of the aerogel taken as 100% by weight. In most embodiments, the etched-carbon polymer aerogel comprises about 78% to about 92% by weight carbon, preferably about 82% to about 88% by weight carbon, and more preferably about 84% to about 86% by weight carbon, based upon the total weight of the aerogel taken as 100% by weight. Furthermore, the etched-carbon polymer aerogel comprises about 0.1% to about 3% by weight hydrogen, preferably about 0.5% to about 2.5% by weight hydrogen, and more preferably about 1% to about 2% by weight hydrogen, based upon the total weight of the aerogel taken as 100% by weight. Additionally, the etched-carbon polymer aerogel comprises about 1% to about 6% by weight nitrogen, preferably about 2% to about 4.5% by weight nitrogen, and more preferably about 2.5% to about 4% by weight nitrogen, based upon the total weight of the aerogel taken as 100% by weight. The etched-carbon polymer aerogel comprises about 5% to about 13% by weight oxygen, preferably about 7.5% to about 11.5% by weight oxygen, and more preferably about 8% to about 10% by weight oxygen, based upon the total weight of the aerogel taken as 100% by weight.

To synthesize the etched-carbon polymer aerogels, a fully oxidized polymer aerogel or carbon polymer aerogel, preferably a carbon polymer aerogel, and more preferably a carbon polymer aerogel processed above 700° C., is etched by first subjecting it to heat treatment under argon or nitrogen gas, preferably argon, at a flow rate of from about 0.05 L min⁻¹ to about 1 L min⁻¹, preferably about 0.09 L min⁻¹ to about 0.7 L min⁻¹. While subjected to argon, the fully oxidized polymer aerogel or carbon polymer aerogel is heated at a temperature of from about 800° C. to about 1,200° C., preferably from about 850° C. to about 1,100° C., more preferably from about 950° C. to about 1,150° C., and even more preferably about 1,000° C. at a heating rate of from about 1° C. min⁻¹ to about 4° C. min⁻¹. Once the desired temperature is reached, the fully oxidized polymer aerogel or carbon polymer aerogel is exposed to an etchant, such as CO₂, at a flow rate of from about 0.05 L min⁻¹ to about 1 L min⁻¹, preferably about 0.09 L min⁻¹ to about 0.7 L min⁻¹ for a time period of about 1 hour to about 5 hours, preferably about 2 hours to about 4 hours, more preferably about 2.5 to about 3.5 hours, and even more preferably 3 hours. After this time period, the fully oxidized polymer aerogel or carbon polymer aerogel is subjected once more to argon and cooled to room temperature at a heating rate of from about 1° C. min⁻¹ to about 4° C. min⁻¹.

### Properties

It will be appreciated that the monolithic polymer aerogels possess properties not previously achieved. Because the nitrogen in the polymer aerogels has been introduced by an in-situ method (i.e., the polymer aerogels are formed from nitrogen-containing precursors), the aerogels are generally more stable and have higher surface areas and pore volumes than if the aerogels had been treated with nitrogen containing molecules after production.

Furthermore, each type of polymer aerogel possesses its own unique set of properties that make each ideal for different purposes. For instance, the partially oxidized polymer aerogels exhibit a Brunauer-Emmett-Teller (BET) multipoint surface area of from about 5 m²/g to about 30 m²/g, preferably about 10 m²/g to about 25 m²/g, and more preferably about 15 m²/g to about 20 m²/g, and have an average micropore surface area of from about 1 m²/g to about 5 m²/g, preferably about 1.5 m²/g to about 3.5 m²/g, and more preferably about 2 m²/g to about 3 m²/g. The partially oxidized polymer aerogels also have a bulk density of from about 0.07 g/cm³ to about 0.7 g/cm³, preferably about 0.09 g/cm³ to about 0.5 g/cm³, and more preferably about 0.1 g/cm³ to about 0.3 g/cm³, and have a skeletal density of from about 1 g/cm³ to about 2.4 g/cm³, preferably about 1.2 g/cm³ to about 2.2 g/cm³, and more preferably about 1.4 g/cm³ to about 1.8 g/cm³. In addition, the partially oxidized polymer aerogels have a porosity of from about 82% to about 94%, preferably about 84% to about 92%, and more preferably about 86% to about 90%. The partially oxidized polymer aerogels also have a specific pore volume of from about 3.9 cm³/g to about 5.1 cm³/g, preferably about 4.1 cm³/g to about 4.9 cm³/g, and more preferably about 4.3 cm³/g to about 4.7 cm³/g, and have an average pore diameter of from about 1,000 nm to about 1,300 nm, preferably about 1,050 nm to about 1,250 nm, and more preferably about 1,100 nm to about 1,200 nm. Finally, the partially oxidized polymer aerogels have a linear shrinkage of from about 15% to about 35%, preferably about 20% to about 30%, and more preferably about 22% to about 28%.

The fully oxidized polymer aerogels exhibit a BET multipoint surface area of about 30 m²/g to about 60 m²/g, preferably about 35 m²/g to about 55 m²/g, and more preferably about 40 m²/g to about 50 m²/g, and have an average micropore surface area of from about 3 m²/g to about 6 m²/g, preferably about 3.5 m²/g to about 5.5 m²/g, and more preferably about 4 m²/g to about 5 m²/g. The fully oxidized polymer aerogels also have a bulk density of from about 0.06 g/cm³ to about 0.8 g/cm³, preferably about 0.08 g/cm³ to about 0.6 g/cm³, and more preferably about 0.1 g/cm³ to about 0.4 g/cm³, and have a skeletal density of from about 0.9 g/cm³ to about 2.1 g/cm³, preferably about 1.1 g/cm³ to about 1.9 g/cm³, and more preferably about 1.3 g/cm³ to about 1.7 g/cm³. In addition, the fully oxidized polymer aerogels have a porosity of from about 77% to about 89%, preferably about 79% to about 87%, and more preferably about 81% to about 85%. The fully oxidized polymer aerogels also have a specific pore volume of from about 2.9 cm³/g to about 4.1 cm³/g, preferably about 3.1 cm³/g to about 3.9 cm³/g, and more preferably about 3.3 cm³/g to about 3.7 cm³/g, and have an average pore diameter of from about 200 nm to about 500 nm, preferably about 250 nm to about 450 nm, and more preferably about 300 nm to about 400 nm. Finally, the fully oxidized polymer aerogels have a linear shrinkage of from about 32% to about 46%, preferably about 34% to about 44%, and more preferably about 36% to about 42%.

The carbon polymer aerogels processed below 500° C. exhibit a BET multipoint surface area of from about 10 m²/g to about 50 m²/g, preferably about 15 m²/g to about 45 m²/g, and more preferably about 20 m²/g to about 40 m²/g, and have an average micropore surface area of from about 0.5 m²/g to about 11 m²/g, preferably about 1.5 m²/g to about 10 m²/g, and more preferably about 2 m²/g to about 9.5 m²/g. The carbon polymer aerogels processed below 500° C. also have a bulk density of from about 0.07 g/cm³ to about 0.7 g/cm³, preferably about 0.09 g/cm³ to about 0.5 g/cm³, and more preferably about 0.2 g/cm³ to about 0.3 g/cm³, and have a skeletal density of from about 0.9 g/cm³ to about 2.1 g/cm³, preferably about 1.1 g/cm³ to about 1.9 g/cm³, and more preferably about 1.2 g/cm³ to about 1.7 g/cm³. In addition, the carbon polymer aerogels processed below 500° C. have a porosity of from about 77% to about 89%, preferably about 79% to about 87%, and more preferably about 81% to about 85%. The carbon polymer aerogels processed below 500° C. also have a specific pore volume of from about 3.1 cm³/g to about 4.1 cm³/g, preferably about 2.9 cm³/g to about 3.9 cm³/g, and more preferably about 3.1 cm³/g to about 3.7 cm³/g, and have an average pore diameter of from about 250 nm to about 650 nm, preferably about 300 nm to about 600 nm, and more preferably about 350 nm to about 550 nm. Finally, the carbon polymer aerogels processed below 500° C. have a linear shrinkage of from about 22% to about 36%, preferably about 24% to about 34%, and more preferably about 26% to about 32%.

The carbon polymer aerogels processed between 500° C. and 700° C. exhibit a BET multipoint surface area of from about 200 m²/g to about 500 m²/g, preferably about 275 m²/g to about 375 m²/g, and more preferably about 300 m²/g to about 350 m²/g, and have an average micropore surface area of from about 200 m²/g to about 300 m²/g, preferably about 225 m²/g to about 275 m²/g, and more preferably about 250 m²/g to about 260 m²/g. The carbon polymer aerogels processed between 500° C. and 700° C. also have a bulk density of from about 0.07 g/cm³to about 0.7 g/cm³, preferably about 0.09 g/cm³ to about 0.5 g/cm³, and more preferably about 0.1 g/cm³ to about 0.3 g/cm³, and have a skeletal density of from about 1 g/cm³ to about 0.9 g/cm³ to about 2.1 g/cm³, preferably about 1.1 g/cm³ to about 1.9 g/cm³. In addition, the carbon polymer aerogels processed between 500° C. and 700° C. have a porosity of from about 80% to about 92%, preferably about 82% to about 90%, and more preferably about 84% to about 88%. The carbon polymer aerogels processed between 500° C. and 700° C. also have a specific pore volume of from about 3.4 cm³/g to about 4.6 cm³/g, preferably about 3.6 cm³/g to about 4.4 cm³/g, and more preferably about 3.8 cm³/g to about 4.2 cm³/g, and have an average pore diameter of from about 10 nm to about 70 nm, preferably about 20 nm to about 60 nm, and more preferably about 30 nm to about 50 nm. Finally, the carbon polymer aerogels processed between 500° C. and 700° C. have a linear shrinkage of from about 20% to about 50%, preferably about 25% to about 45%, and more preferably about 30% to about 40%.

The carbon polymer aerogels processed above 700° C. exhibit a BET multipoint surface area of from about 200 m²/g to about 400 m²/g, preferably about 275 m²/g to about 375 m²/g, and more preferably about 300 m²/g to about 350 m²/g, and have an average micropore surface area of from about 200 m²/g to about 300 m²/g, preferably about 225 m²/g to about 275 m²/g, and more preferably about 250 m²/g to about 260 m²/g. The carbon polymer aerogels processed above 700° C. also have a bulk density of about 0.06 g/cm³ to about 0.8 g/cm³, preferably about 0.08 g/cm³to about 0.6 g/cm³, and more preferably about 0.1 g/cm³ to about 0.4 g/cm³, and have a skeletal density of from about 1.6 g/cm³ to about 2.8 g/cm³, preferably about 1.8 g/cm³ to about 2.6 g/cm³, and more preferably about 2 g/cm³ to about 2.4 g/cm³. In addition, the carbon polymer aerogels processed above 700° C. have a porosity of from about 84% to about 96%, preferably about 86% to about 94%, and more preferably about 88% to about 92%. The carbon polymer aerogels processed above 700° C. also have a specific pore volume of from about 2.9 cm³/g to about 4.1 cm³/g, preferably about 3.1 cm³/g to about 3.9 cm³/g, and more preferably about 3.3 cm³/g to about 3.7 cm³/g, and have an average pore diameter of from about 10 nm to about 70 nm, preferably about 20 nm to about 60 nm, and more preferably about 30 nm to about 50 nm. Finally, the carbon polymer aerogels processed above 700° C. have a linear shrinkage of from about 65% to about 77%, preferably about 67% to about 75%, and more preferably about 69% to about 73%.

The etched-carbon polymer aerogels exhibit a BET multipoint surface area of from about 1,500 m²/g to about 1,800 m²/g, preferably about 1,550 m²/g to about 1,750 m²/g, and more preferably about 1,600 m²/g to about 1,700 m²/g, and have an average micropore surface area of from about 500 m²/g to about 900 m²/g, preferably about 600 m²/g to about 800 m²/g, and more preferably about 650 m²/g to about 750 m²/g. In another embodiment, the etched-carbon polymer aerogels have an average micropore surface area of from about 900 m²/g to about 1,200 m²/g, preferably about 950 m²/g to about 1,150 m²/g, and more preferably about 1,000 m²/g to about 1,100 m²/g. The etched-carbon polymer aerogels also have a bulk density of from about 0.06 g/cm³ to about 0.7 g/cm³, preferably about 0.08 g/cm³ to about 0.5 g/cm³, and more preferably about 0.1 g/cm³ to about 0.3 g/cm³, and have a skeletal density of from about 1.2 g/cm³ to about 2.4 g/cm³, preferably about 1.4 g/cm³ to about 2.2 g/cm³, and more preferably about 1.6 g/cm³ to about 2 g/cm³. In addition, the etched-carbon polymer aerogels have a porosity of from about 86% to about 98%, preferably about 88% to about 96%, and more preferably about 90% to about 94%. The etched-carbon polymer aerogels also have a specific pore volume of from about 5.5 cm³/g to about 6.7 cm³/g, preferably about 5.7 cm³/g to about 6.5 cm³/g, and more preferably about 5.9 cm³/g to about 6.3 cm³/g, and have an average pore diameter of from about 1 nm to about 30 nm, preferably about 5 nm to about 25 nm, and more preferably about 10 nm to about 20 nm. Finally, the etched-carbon polymer aerogels have a linear shrinkage of from about 80% to about 92%, preferably about 82% to about 90%, and more preferably about 84% to about 88%.

The BET multipoint surface area (σ) for each type of polymer aerogel is calculated from the medium pressure N₂-sorption isotherm for the aerogel using the Brunauer-Emmett-Teller method, and the average micropore surface area is calculated from a t-plot analysis of the N₂-sorption isotherm for the aerogel using the Harkins and Jura model. Furthermore, the bulk density (ρ_{b}) for each type of polymer aerogel is calculated by dividing the aerogel's weight by its physical dimensions (i.e, length×width×height), and the skeletal density (ρₛ) for each type of polymer aerogel is determined using helium pycnometry with a Micromeritics AccuPyc II 1340 instrument. The porosity (Π) for each type of polymer aerogel is then calculated using the following equation: Π=100×(ρₛ−ρ_{b})/ρₛ. The specific pore volume (V) for each type of polymer aerogel is calculated using the following equation: V_{Total}=(1/ρ_{b})−(1/ρₛ), and the average pore diameter for each type of polymer aerogel is calculated using the following equation: 4×V/σ, where V=V_{Total}. Finally, the linear shrinkage for each type of polymer aerogel is calculated by dividing the length of the resulting aerogel by its original length.

### Methods of Using the Polymer Aerogels

Though the above-described polymer aerogels can be used in many applications (e.g., thermal and acoustic insulation, electronic devices, capacitors, imaging devices, catalysts, pesticides, and cosmic dust collection), the aerogels are preferably used to sorb CO₂ from the environment, particularly used for pre-combustion CO₂ capture from the air and/or post-combustion CO₂ capture from flue gases. As used herein, the terms “sorb” and “sorption” mean to uptake or hold by adsorption, absorption, or a combination of adsorption and absorption. It will be appreciated that the polymer aerogels, preferably the carbon and the etched-carbon polymer aerogels, exhibit high CO₂ sorption (preferably adsorption) capacity at standard temperature and pressure (i.e., 273 K (or 0° C.) at 1 bar) and/or at standard ambient temperature and pressure (i.e., 298 K (or 25° C.), 1 bar). Particularly, the carbon polymer aerogels have a CO₂ sorption capacity of about 2 mmol/g to about 8 mmol/g, preferably about 3 mmol/g to about 7 mmol/g, and more preferably about 4 mmol/g to about 6 mmol/g. Additionally, the etched-carbon polymer aerogels have a CO₂ sorption capacity of at least about 7 mmol/g, preferably at least about 7.5 mmol/g, and more preferably at least about 8 mmol/g. In other embodiments, the etched-carbon polymer aerogels have a CO₂ sorption capacity of about 6 mmol/g to about 16 mmol/g, preferably about 8 mmol/g to about 14 mmol/g, and more preferably about 10 mmol/g to about 12 mmol/g. The foregoing results are generally achieved by placing the polymer aerogel in a CO₂-containing environment for about 30 minutes to about 4 hours.

Without being bound to any particular theory, it was found that this superior property is a result of pore filling beyond monolayer coverage starting with preferential interaction of CO₂ with surface pyridinic and pyridonic nitrogen to form carbamate in a near energy-neutral reaction. The proposed mechanism of CO₂ uptake proceeds as shown in Scheme 5A.

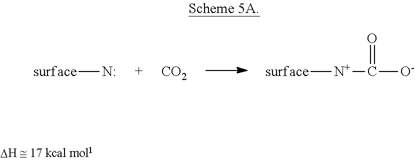

Surface (—O—) is engaged in a neutral reaction, which forms carbonate and continues until all of the micropores are filled as shown in Scheme 5B.

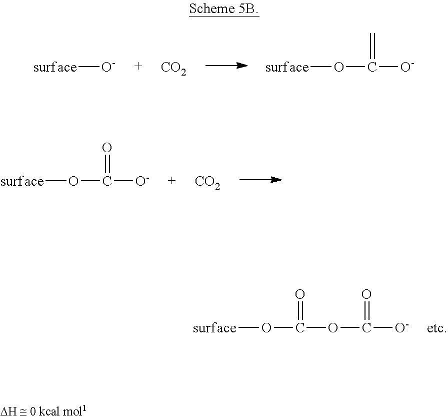

It will also be appreciated that the polymer aerogels, preferably the carbon and the etched-carbon polymer aerogels, exhibit high water sorption (preferably adsorption) capacity. Particularly, the carbon polymer aerogels have a water sorption capacity of about 25% w/w to about 60% w/w, preferably about 30% w/w to about 55% w/w, more preferably about 35% w/w to about 50% w/w, and even more preferably about 42% w/w. Additionally, the etched-carbon polymer aerogels have a water sorption capacity of about 95% w/w to about 135% w/w, preferably about 100% w/w to about 130% w/w, more preferably about 105% w/w to about 125% w/w, and even more preferably about 115% w/w. As a result, the aerogels may also be used as high-capacity desiccants or as water-harvesting tools.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration, and nothing therein should be taken as a limitation upon the overall scope of the invention.

### Example 1

### Materials

The materials used in the following Examples were obtained from the sources described in this paragraph. Anthranilic acid, formaldehyde, aniline, triethyl orthoformate, aluminum chloride, lithium aluminum hydride and tetrahydrofuran (THF) were obtained from the Sigma Aldrich Chemical Company (St. Louis, MO). ¹⁵N-enriched anthranilic acid and ¹⁵N-enriched aniline were purchased from Cambridge Isotope Limited (Tewksbury, MA). Dimethylformamide (DMF), concentrated hydrochloric acid (HCl), triphosgene, dioxane, ethanol, methanol, and acetone were obtained from Fischer Scientific International, Inc. (Hampton, NH). Siphon grade CO₂, argon (99.999%), N₂ (99.999%), H₂ (99.999%), and CH₄ were purchased from Ozark Gas (Rolla, MO). Liquid N₂ and O₂ (99.999%) were purchased form Air Gas (St. Louis, MO). A propane hand torch cylinder (87.5-100%) was purchased form BernzOmatic (Newark, NJ). All the reagents and solvents were used as received, unless noted otherwise.

### Example 2

Synthesis of Bis(3-phenyl-1,2, 3, 4-tetrahydroquinazolin-6-yl)methane (THQ)
1. Synthesis of Compound 2: 5,5′-methylenebis(2-aminobenzoic acid) (Also Referred to as 4,4′-methylene bisanthranilic acid)

Referring to Scheme 1, in a 1 neck round bottom flask, 27.4 grams (200 mmol) of compound 1 anthranilic acid was added to 500 mL of H₂O and 100 mL of 36% HCl, and the mixture was magnetically stirred and heated at 50° C. with an oil bath for 10-15 minutes. Then, 140 mL (114 mmol) of a (3% w/w) aqueous formaldehyde solution was added to the flask. The resulting solution was heated at 70° C. and stirred for 4 hours. At the end of the period, the solution was allowed to cool to room temperature and was neutralized with concentrated aqueous ammonium hydroxide to a pH of approximately 7, forming a precipitate. The precipitate was collected using a filter (FisherbrandTM Fluted Qualitative Filter Paper Circles), washed with hot H₂O, and air-dried on the filter for 16 hours. The process yielded 45.5 grams of compound 2 (melting point: 258-260° C.) as a yellow solid (yield=79.5% w/w) having the following chemical structure:

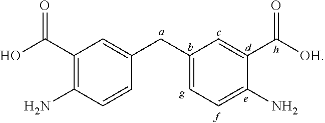 [SMILES: Nc1ccc(Cc2ccc(N)c(C(=O)O)c2)cc1C(=O)O]

¹H NMR (400 MHZ, DMSO-d₆), ¹³C NMR (100 MHz, DMSO-d₆), attenuated total reflectance infrared spectra (ATR-FTIR), and ¹³C NMR Attached Proton Test (APT) (100 MHZ, DMSO-d₆) were used to confirm the synthesis of compound 2. ¹H NMR (400 MHZ, DMSO-d₆): δ (ppm) 8.50 (bs, 4H, NH₂), 7.47 (s, 2H, H_{c}), 7.04 (d, J=8.4 Hz, 2H, H_{g}), 6.67 (d, J=8.4 Hz, 2H, H_{f}), 3.60 (s, 2H, Hₐ). ¹³C NMR (100 MHZ, DMSO-d₆): δ (ppm) 169.5 (s, 2C, Cₕ), 149.7 (s, 2C, Cₑ), 134.4 (s, 2C, C_{g}), 130.5 (s, 2C, C_{c}), 127.7 (s, 2C, C_{b}), 116.6 (s, 2C, C_{f}), 109.4 (s, 2C, C_{d}), 39.0 (s, 1C, Cₐ). ¹³C NMR APT chemical shift values are same as ¹³C NMR chemical shift values, except that the shifts are in the opposite direction due to the number of hydrogens bonded to each carbon. ATR-FTIR (cm⁻¹): 3472 (NH₂ stretching), 3370 (NH₂ stretching), 3071 (OH stretching), 2923, 1664 (C═O stretching), 1584, 1557, 1490, 1409, 1297, 1229, 1204, 1165, 1102, 892, 837, 675. CHN elemental analysis (% w/w), calculated for C₁₅H₁₄N₂O₄: C, 62.93, H, 4.93, N, 9.79, O, 22.35 (% O was calculated by the following equation: [100−(% C+% H+% N)]). Found: C, 62.04, H, 4.88, N, 9.82, O, 23.26.

2. Synthesis of Compound 3: 6,6′-methylenebis(1H-benzo[d][ 1,3 ]oxazine-2,4-dione)

To synthesize compound 3, 28.6 grams (100 mmol) of compound 2 synthesized in Part 1 and 19.5 grams (66 mmol) of triphosgene were added to 400 mL of dioxane. The mixture was heated to reflux at 100° C. for 6 hours, forming a precipitate. The precipitate was collected, washed with hot dioxane, and was dried in a vacuum oven at 80° C. for 12 hours. The process yielded 32.2 grams of compound 3 (melting point: 260° C. (dec.)) as a white solid (yield=95.2% w/w) having the following chemical structure:

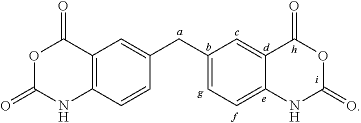 [SMILES: O=c1[nH]c2ccc(Cc3ccc4[nH]c(=O)oc(=O)c4c3)cc2c(=O)o1]

¹H NMR (400 MHZ, DMSO-d₆), ¹³C NMR (100 MHZ, DMSO-d₆), ATR-FTIR, and ¹³C NMR APT (100 MHz, DMSO-d₆) were used to confirm the synthesis of compound 3. ¹H NMR (400 MHZ, DMSO-d₆): δ (ppm) 11.6 (s, 2H, NH), 7.80 (s, 2H, H_{c}), 7.65 (d, J=8.4 Hz, 2H, H_{g}), 7.10 (d, J=8.4 Hz, 2H, H_{f}), 4.04 (s, 2H, Hₐ). ¹³C NMR (100 MHZ, DMSO-d₆): δ (ppm) 160.0 (s, 2C, Cₕ), 147.1 (s, 2C, Cᵢ), 139.9 (s, 2C, Cₑ), 137.6 (s, 2C, C_{b}), 136.4 (s, 2C, C_{g}), 128.4 (s, 2C, C_{c}), 115.7 (s, 2C, C_{f}), 110.4 (s, 2C, Cₐ), 38.7 (s, 1C, Cₐ). ¹³C NMR APT chemical shift values are same as ¹³C NMR chemical shift values, except that the shifts are in the opposite direction due to the number of hydrogens bonded to each carbon. ATR-FTIR (cm⁻¹): 3176 (NH stretching), 3087, 2980, 2927, 1776 (C═O stretching), 1731 (C—O stretching), 1625, 1514, 1431, 1338, 1271, 1255, 1112, 1080, 1032, 985, 861, 746. CHN elemental analysis (% w/w), calculated for C₁₇H₁₀N₂O₆: C, 60.36, H, 2.98, N, 8.28, 0, 28.38 (% O was calculated by the following equation: [100−(% C+% H+% N)]). Found: C, 60.40, H, 2.94, N, 8.32, O, 28.34.

3. Synthesis of Compound 4: 6,6′-methylenebis(3-phenylquinazolin-4(3H)-one)

To synthesize compound 4, 27.9 g of aniline (300 mmol) was added to a mixture of 33.8 grams (100 mmol) of compound 3 prepared using the method described in Part 2 of this Example and 44.4 grams (300 mmol) of triethyl orthoformate in 500 mL of ethanol. The resulting mixture was magnetically stirred at 80° C. for 18 hours. The mixture was allowed to cool to room temperature, forming a precipitate. The precipitate was collected, washed with ethanol, and air-dried in a vacuum oven at 80° C. for 12 hours. The process yielded 30.6 grams of compound 4 (melting point: 208-210° C.) as a white solid (yield=66.9% w/w) having the following chemical structure:

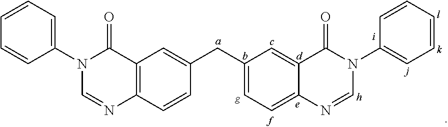 [SMILES: O=c1c2cc(Cc3ccc4ncn(-c5ccccc5)c(=O)c4c3)ccc2ncn1-c1ccccc1]

¹H NMR (400 MHZ, CDCl₃), ¹³C NMR (100 MHz, CDCl₃), ATR-FTIR, and ¹³C NMR APT (100 MHZ, CDCl₃) were used to confirm the synthesis of compound 4. ¹H NMR (400 MHZ, CDCl₃): δ (ppm) 8.19 (s, 2H, Hₕ), 8.07 (s, 2H, H_{c}), 7.70 (d, J=8.4 Hz, 2H, H_{g}), 7.62 (d, J=8.4 Hz, 2H, Hᵣ), 7.52 (t, J=7.2, 4H, Hₖ), 7.46 (t, J=7.2, 2H, Hₗ), 7.40 (d, J=7.2, 4H, Hⱼ), 4.27 (s, 2H, Hₐ). ¹³C NMR (100 MHZ, CDCl₃): δ (ppm) 161.1 (s, 2C, C═O), 147.1 (s, 2C, Cₕ), 146.3 (s, 2C, Cₑ), 140.6 (s, 2C, Cᵢ), 138.0 (s, 2C, C_{b}) 136.1 (s, 2C, C_{g}), 130.1 (s, 4C, Cₖ), 129.6 (s, 2C, C_{c}), 128.5 (s, 4C, Cₗ), 127.5 (s, 2C, Cⱼ), 127.3 (s, 2C, C_{f}), 122.9 (s, 2C, C_{d}), 41.9 (s, 1C, Cₐ). ¹³C NMR APT chemical shift values are same as ¹³C NMR chemical shift values, except that the shifts are in the opposite direction due to the number of hydrogens bonded to each carbon. ATR-FTIR (cm⁻¹): 3053, 3033, 1773, 1698 (C═O stretching), 1593, 1484, 1453, 1336, 1260, 1025, 986, 748. CHN elemental analysis (% w/w), calculated for C₂₉H₂₀N₄O₂: C, 76.30, H, 4.42, N, 12.27, 0, 7.01 (% O was calculated by the following equation: [100−(% C+% H+% N)]). Found: C, 76.22, H, 4.40, N, 12.22, 0, 7.16.

### 4. Synthesis of Compound THQ

To synthesize compound THQ, 10.6 grams (80 mmol) of aluminum chloride was added to 320 mL of tetrahydrofuran (THF), and this solution was added to a suspension comprising 4.56 grams (120 mmol) of lithium aluminum hydride in 280 mL of THF. The resulting mixture was stirred at 0° C. for 1 hour. Then, 18.2 grams (40 mmol) of compound 4 synthesized in Part 3 above was added as a solid to this cold solution, and this mixture was stirred at 0° C. for 2 hours. At the end of the period, stirring was stopped and crushed ice was added to the mixture. The solids produced were filtered off and washed with cold THF. The mother liquid and the THF washes were combined, and the solvent was removed under reduced pressure (5 KPa to 6 KPa) with a rotary evaporator 35° C. to 45° C. for 30 minutes to 45 minutes. to afford the crude product as a yellow solid (14.6 grams, yield=84% w/w). Subsequently, in a round-bottom flask, the crude product was heated to reflux at 65° C. to 70° C. for 2 hours in 230 mL of methanol. The resulting solid was filtered hot, washed with hot methanol, and dried under vacuum at 80° C. for 12 hours to 16 hours. The process yielded 7.5 grams of compound THQ (melting point: 150-153° C.) as a white solid (yield=43% w/w) having the following chemical structure:

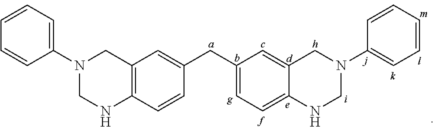 [SMILES: c1ccc(N2CNc3ccc(Cc4ccc5c(c4)CN(c4ccccc4)CN5)cc3C2)cc1]

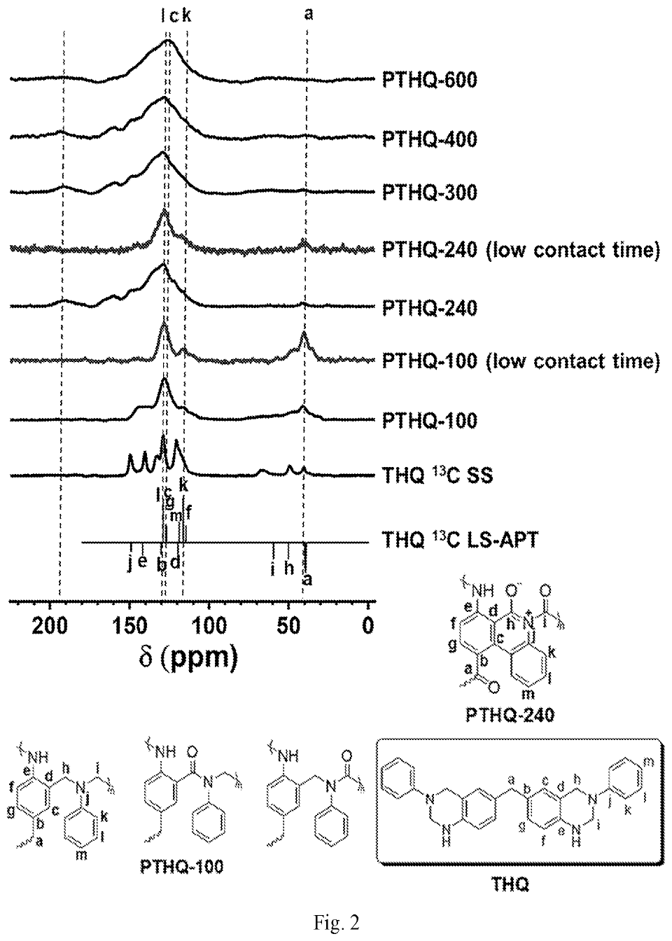

¹H NMR (400 MHZ, DMSO-d₆), ¹³C NMR (100 MHZ, DMSO-d₆), ¹⁵N NMR, Solid-state ¹⁵N NMR, ATR-FTIR, ¹³C NMR APT (100 MHZ, DMSO-d₆) (shown in FIGS. 2), and ¹⁵N-¹H HETCOR NMR were used to confirm the synthesis of compound THQ. ¹H NMR (400 MHZ, DMSO-d₆): δ˜ (ppm) 7.20 (m, 4H, Hₗ), 7.02 (d, J=8.2 Hz, 4H, Hₖ), 6.78 (m, 6H, H_{c}, H_{g}, Hₘ) 6.42 (d, J=8.2 Hz, 2H, Hᵣ), 5.82 (t, J=3.5, 2H, NH), 4.60 (d, J=3.5 Hz, 4H, Hᵢ), 4.41 (s, 4H, Hₕ), 3.58 (s, 2H, Hₐ). ¹³C NMR (100 MHZ, DMSO-d₆): δ (ppm) 149.0 (s, 2C, Cⱼ), 141.8 (s, 2C, C_{c}), 130.3 (s, 2C, C_{b}), 128.9 (s, 4C, Cₗ), 127.2 (s, 2C, C_{c}), 126.8 (s, 2C, C_{g}), 119.6 (s, 2C, C_{d}), 118.8 (s, 2C, Cₘ), 116.3 (s, 4C, Cₖ), 114.7 (s, 2C, C_{f}), 59.6 (s, 2C, Cᵢ), 50.1(s, 2C, Cₕ), 39.9 (s, 1C, Cₐ). ¹³C NMR APT chemical shift values are the same as the ¹³C NMR chemical shift values, except that the shifts are in the opposite direction due to the number of hydrogens bonded to each carbon. ¹⁵N NMR (40.557 MHz, DMF-d₇): δ (ppm) 60.09. Solid-state ¹⁵N NMR (40.557 MHz; magic angle spinning 5 kHz): δ (ppm) 67.13, 59.48. ¹⁵N-¹H NMR: δ (ppm) δN/δH 60.09/5.82. ATR-FTIR (cm⁻¹): 3252 (NH stretching), 3014, 2888, 2830, 1596 (C—C stretching), 1491 (tri-substituted benzene stretching), 1450, 1298 (C—N stretching), 1248, 1219, 1125, 1028, 992, 926, 825, 745, 689. CHN elemental analysis (% w/w), calculated for C₂₉H₂₈N₄: C, 80.52, H, 6.53, N, 12.95. Found: C, 80.68, H, 6.36, N, 12.96.

### Example 3

### Synthesis of PTHQ-100, PTHQ-240, C-PTHQ-800, and EC-PTHQ-1000 Aerogels

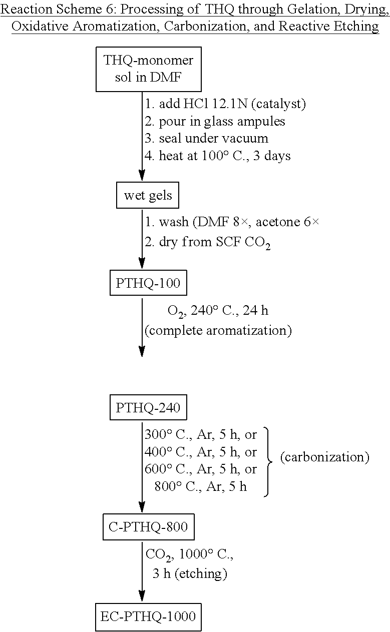

### 1. Methods

The pyrolytic processes described in Parts 4-6 of this Example were conducted in a tube furnace (MTI GSL1600X-80). The heating rate was always 2.5° C. min⁻¹, and the gas (argon, oxygen, or CO₂) flow rate was always set at 0.3 L min⁻¹.

### 2. Preparation of PTHQ-100 Polymer

To prepare the PTHQ-100 polymer, 10 grams of the THQ monomer prepared according to the method described in Part 4 of Example 2 was dissolved in 38 grams of DMF. Then, 2 grams of concentrated (aq) HCl (12.1 N) was added to this solution to obtain the sol. The weight percent concentration of the THQ monomer in the sol was 20% w/w. The sol was stirred at room temperature for 10 minutes and then was syringed into flame-elongated Pyrex™ tubes (1 cm diameter×10 cm length), which were used as molds. Each tube was purged with argon for 1 minute and subsequently was degassed with three freeze-pump-thaw cycles at 77 K. At the end of the third cycle, and while tubes remained submerged in liquid nitrogen, the tubes were flame-sealed under vacuum using a propane hand-held torch (BernzOmatic, TS3500K). The sol inside the vacuum-sealed tubes was left for gelation at 100° C. for 3 days. At the end of the period, the tubes were broken open, and the gels were removed and washed 8 times (4 hours each time) with DMF and 6 times (4 hours each time) with acetone. The amount of solvent used for each wash was four times the volume of the gel.

### 3. Synthesis of PTHQ-100 Aerogels

The wet gels prepared in Part 2 above were dried in an autoclave (SPIDRY Jumbo Supercritcal Point Dryer, SPI Supplies, Inc. West Chester, PA). The wet gel samples were placed in a special boat inside the autoclave, and acetone was added to the boat until the samples were submerged. The boat was then loaded to the autoclave, which was kept at 14° C. After the boat was loaded, the pressure vessel was closed. Liquid CO₂ was allowed in multiple times, and each time the liquid CO₂ was drained out through a valve at the bottom of the autoclave. This cycle was repeated until all the acetone was extracted out of the pores of the samples completely. The end-of-process criterion was that CO₂ released through the drain valve formed dry ice. Subsequently, the temperature of the autoclave was raised to 40° C. for 1 hour. After a stay at that temperature for 1 hour, supercritical fluid (SCF) CO₂ was vented off as a gas over a period of 5 hours. The resulting aerogels are referred to as PTHQ-100 (yield=42.23% w/w).

### 4. Oxidative Aromatization of PTHQ-100 to PTHQ-240

The PTHQ-100 aerogel monoliths prepared in Part 3 above were placed in a tube furnace under flowing O₂ (0.3 L min⁻¹) and were heated to 240° C. (2.5° C. min⁻¹) for 24 hours. The resulting aerogels are referred to as PTHQ-240 (yield=99.95% w/w).

### 5. Pyrolytic Conversion of PTHQ-240 Aerogels to PTHQ-300, PTHQ-400, PTHQ-600, or C-PTHQ-800 Aerogels

The PTHQ-240 aerogels prepared according to Part 4 above were placed in the tube furnace under flowing argon (0.3 L min⁻¹) and were heated at 800° C. (2.5° C. min⁻¹) for 5 hours. The resulting aerogels are referred to as C-PTHQ-800 (yield=60.69% w/w) due to the temperature used for pyrolysis. For characterization purposes along pyrolysis, several PTHQ-240 aerogel monoliths were heated for 5 hours at each of the following temperatures: 300° C. (yield=91.34% w/w), 400° C. (yield=88.56% w/w), and 600° C. (yield=67.07% w/w). These aerogels are referred to as PTHQ-X, where X represents the temperature used for pyrolysis. All samples were returned to room temperature and were characterized.

### 6. Reactive Etching of C-PTHQ-800 Aerogels to EC-PTHQ-1000 Aerogels

The C-PTHQ-800 aerogels prepared in Part 5 above were placed in the tube furnace under flowing argon (0.3 L min⁻¹) and were heated at 1000° C. (2.5° C. min⁻¹). Then, the flowing gas was switched to CO₂ (0.3 L min⁻¹) for 3 hours, and then back to argon. The tube furnace was cooled back to room temperature at 2.5° C. min⁻¹, and the resulting etched-carbon aerogels are referred to as EC-PTHQ-1000 (yield=34.67% w/w).

### Example 4

Synthesis of ¹⁵N-enriched THQ, ¹⁵N-enriched PTHQ-100 Aerogels, and ¹⁵N-enriched PTHQ-240 Aerogels

### 1. Synthesis of ¹⁵N-enriched Anthranilic Acid THQ

To synthesize ¹⁵N-enriched anthranilic acid THQ (THQ with only its secondary nitrogen position enriched with ¹⁵N), the processes described in Example 2 above were used (on a reduced scale), except, in Part 1, anthranilic acid was replaced with a mixture of ¹⁵N-enriched and regular anthranilic acid (containing 10% mol:mol of the enriched compound).

The process yielded 3 grams of ¹⁵N-enriched anthranilic acid THQ (melting point: ˜150-153° C.) (yield=43% w/w). Solid-state ¹⁵N NMR (¹⁵N NMR) was used to confirm the synthesis of ¹⁵N-enriched anthranilic acid THQ. ¹⁵N NMR (40.557 MHz; 5,000 Hz): δ (ppm) 66.87, 59.48.

### 2. Synthesis of ₁₅N-enriched Aniline THQ

To synthesize ₁₅N-enriched aniline THQ (THQ with only its tertiary nitrogen position enriched with ₁₅N), the processes described in Example 2 above were used (on a reduced scale), except, in Part 3, aniline was replaced with a mixture of ¹⁵N-enriched and regular aniline (containing 10% mol:mol of the enriched compound).

The process yielded 3 grams of ¹⁵N-enriched aniline THQ (melting point: ˜150-153° C.). ¹⁵N NMR was used to confirm the synthesis of ¹⁵N-enriched aniline THQ. ¹⁵N NMR (40.557 MHZ; 5,000 Hz): δ (ppm) 67.13.

### 3. Synthesis of ¹⁵N-enriched Anthranilic Acid and Aniline THQ

To synthesize ¹⁵N-enriched anthranilic acid and aniline THQ (THQ with both nitrogen positions enriched with ¹⁵N (in an 1:1 mole ratio)), the processes described in Example 2 above were used (on a reduced scale), except, in Part 1, anthranilic acid was replaced with a mixture of ¹⁵N-enriched and regular anthranilic acid (containing 10% mol:mol of the enriched compound) and, in Part 3, aniline was replaced with a mixture of ¹⁵N-enriched and regular aniline (containing 10% mol:mol of the enriched compound).

The process yielded 3 grams of ¹⁵N-enriched anthranilic acid and aniline THQ (melting point: ˜150-153° C.) (yield=43% w/w). ¹⁵N NMR was used to confirm the synthesis of ¹⁵N-enriched anthranilic acid and aniline THQ. ¹⁵N NMR (40.557 MHz; 5,000 Hz): δ (ppm) 67.35, 59.30.

4. Synthesis of ¹⁵N-enriched PTHQ-100 and ¹⁵N-enriched PTHQ-240 Aerogels

Using the processes described in Parts 2-3 of Example 3, ¹⁵N-enriched anthranilic acid THQ, ¹⁵N-enriched aniline THQ, and ¹⁵N-enriched anthranilic acid and aniline THQ synthesized in Parts 1-3 above were used to make ¹⁵N-enriched anthranilic acid PTHQ-100 aerogels, ¹⁵N-enriched aniline PTHQ-100 aerogels, and ¹⁵N-enriched anthranilic acid and aniline PTHQ-100 aerogels (yield=42.23% w/w).

These ¹⁵N-enriched PTHQ-100 aerogels were aromatized to ¹⁵N-enriched PTHQ-240 aerogels (yield=99.95% w/w) as described in Part 4 of Example 3.

### Example 5

### Thermal Characterization of PTHQ-100 Aerogels

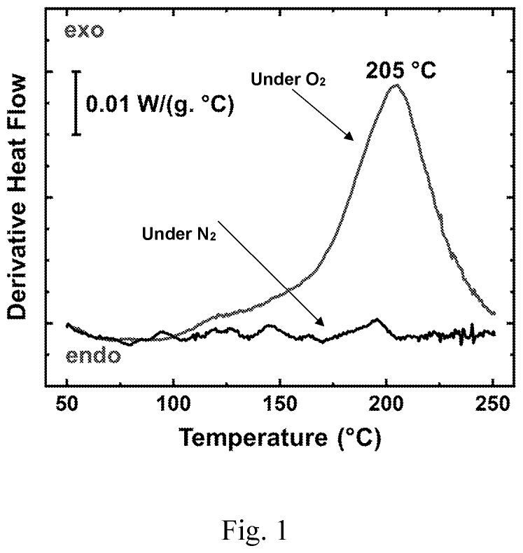

Modulated Differential scanning calorimetry (MDSC) was conducted under N₂ and O₂ with a TA Instruments Differential Scanning calorimetry Model Q2000 calibrated against sapphire standard and run from 35° C. to 350° C. using heating rate of 5° C. min⁻¹. The T4P modulation mode was used with a 60 s modulation period and 1° C. as the modulation amplitude. Notably, as shown in FIG. 1, MDSC of PTHQ-100 aerogels under O₂ showed a major exothermic event at 205° C., which was absent from the MDSC data under N₂.

### Example 6

### Chemical Characterization of Aerogels

### 1. Methods

CHN elemental analysis was conducted with an Exeter Analytical Model CE440 elemental analyzer, which was calibrated with acetanilide, urea, and glycine. The combustion furnace was operated at 1050° C. The calibration standards and samples were run three times, and results were provided as averages.

Attenuated total reflectance infrared spectra (ATR-IR) were obtained with a Nicolet FTIR Model iS50 spectrophotometer. ¹H, ¹³C, APT (Attached Proton Test), and ¹⁵N liquid NMR spectra were recorded with a 400 MHz Varian Unity Inova NMR instrument (100 MHz and 40.557 MHz carbon and nitrogen frequencies, respectively). ¹H-¹⁵N HETCOR NMR spectra were recorded on the same spectrometer [(400 MHZ(¹H), 40.557 MHZ(¹⁵N)].

Solid-state CP TOSS ¹³C NMR spectra were obtained from samples grounded into fine powders on a Bruker Avance III 400 MHz spectrometer with a carbon frequency equal to 100 MHz, using a 7 mm Bruker MAS probe at a magic angle spinning rate of 5 kHz with broadband proton suppression and CP TOSS pulse sequence for total suppression of side spinning bands. Solid-state ¹³C NMR spectra were referenced externally to glycine (carbonyl carbon at 176.03 ppm). Chemical shifts were reported versus tetramethylsilane (TMS, 0 ppm). Solid-state CPMAS ¹⁵N NMR spectra were obtained on the same Bruker Avance III 400 MHz spectrometer with a nitrogen frequency of 40.557 MHz, using a 7 mm Bruker MAS probe at a magic angle spinning at 5 kHz with broadband proton suppression. Chemical shifts were reported versus liquid ammonia (0 ppm) and were externally referenced to glycine (amine nitrogen at 33.40 ppm versus liquid ammonia). In all the ¹³C and ¹⁵N solid-state NMR experiments, the relaxation delay was set at 5 s. In selected ¹⁵N solid-state NMR experiments, results were compared at two different contact times (P15): 5 μs and 3000 μs.

X-Ray photoelectron spectroscopic analysis (XPS) was carried out with a Kratos Axis 165 Photoelectron Spectroscopy System. Samples were mixed and ground together with Au powder (5% w/w) as internal energy reference. Samples were analyzed as powders. Each sample was placed on a piece of conductive carbon tape that was adhered to a stainless-steel sample holder. Samples were introduced into the analysis chamber one at a time, and the chamber was evacuated at ≤10⁻⁸ Torr. No ion sputtering was performed on any of the samples. An Al monochromatic source (150 W) was used for excitation. A charge neutralizer was used to reduce the effects of differential or sample charging. The analysis area was 700×300 μm. Elemental quantification calculations were based on broad survey results from single sweeps at higher sensitivity (pass energy=80) and were carried out with the Kratos Axis Vision processing software, taking into consideration the appropriate relative sensitivity factors for the particular XPS system. High-resolution elemental scans were carried out at a lower sensitivity (pass energy=20), using multiple sweeps to improve the signal-to-noise ratio. Deconvolution of the spectra was performed with Gaussian function fitting, using the OriginPro 9.7 software package.

2. Chemical Characterization of THQ, ¹⁵N-enriched THQ, PTHQ-100, ¹⁵N-enriched PTHQ-100, PTHQ-240, and ¹⁵N-enriched PTHQ-240

The chemical identity of THQ, PTHQ-100, and PTHQ-240 was probed with solid-state CPMAS ¹³C NMR with contact time (P15=3000 μs). For THQ, liquid-state ¹³C APT was used to identify the respective peaks. For PTHQ-100 & PTHQ-240, additional ¹³C NMR with low contact time (P15=5 μs) was used which forced carbons with no Hs to vanish. SS ¹⁵N NMR was used specifically for understanding the mechanism of polymerization and ring-fusion aromatization. Additionally, FTIR and CHN elemental analysis data was used to support the chemical transformation along the polymerization as identified by ¹⁵N NMR.

¹³C APT spectrum and SS ¹³C spectrum of THQ (prepared according to the methods described in Example 2) along with SS ¹³C NMR spectra of PTHQ-100 (prepared according to the methods described in Parts 2-3 of Example 3 and prepared as a sample according to the methods described in Part 1 of this Example) and PTHQ-240 (prepared according to the methods described in Parts 2-4 of Example 3 and prepared as a sample according to the methods described in Part 1 of this Example) are shown in FIG. 2. From the APT data, the resonance at 40 ppm corresponds to the bridge carbon between the aromatic rings in THQ represented by a. Two other peaks in the aliphatic region of THQ at 50 and 60 ppm are represented by h and i, respectively, and correspond to carbons of Mannich bridge. After the HCl catalyzed ring-opening polymerization at 100° C., similar intensity resonance at 40 ppm was observed, confirming the survival of the bridge carbon during the polymerization process. The intensity of h and i became extremely weak after polymerization. In the NMR spectra of PTHQ-240, no significant peak in the aliphatic region was observed, indicating complete oxidation of the bridge carbon and Mannich bridge carbons. This oxidation gave rise to a new resonance at 192 ppm, which could be assigned to the carbonyl on the bridge carbon formed after oxidation. The oxidation on the carbons to form a carbonyl group goes via superoxide/hydroperoxyl chemistry, which results in the formation of carbonyl functionality at the expense of Hs on the carbons. To track the formation of these carbonyls, low contact time experiments for PTHQ-100 and PTHQ-240 were carried out in which carbons with no Hs do not show up. In the low contact time PTHQ-100 spectrum, peak a appeared with significant intensity, indicating no oxidation on the bridging carbon. This result was further confirmed with the absence of carbonyl resonance in PTHQ-100 spectrum. However, peaks h and i had a low intensity, indicating partial oxidation at either position. This partial oxidation creates additional functionality in the polymeric backbone of PTHQ in the form of urea and/or amide. For PTHQ-240, no significant presence of h and i was observed indicating oxidation of Mannich bridge carbons, thus forming urea and amide. This result agrees with the presence of resonance around 160 ppm, which could be assigned to urea/amide carbonyl.

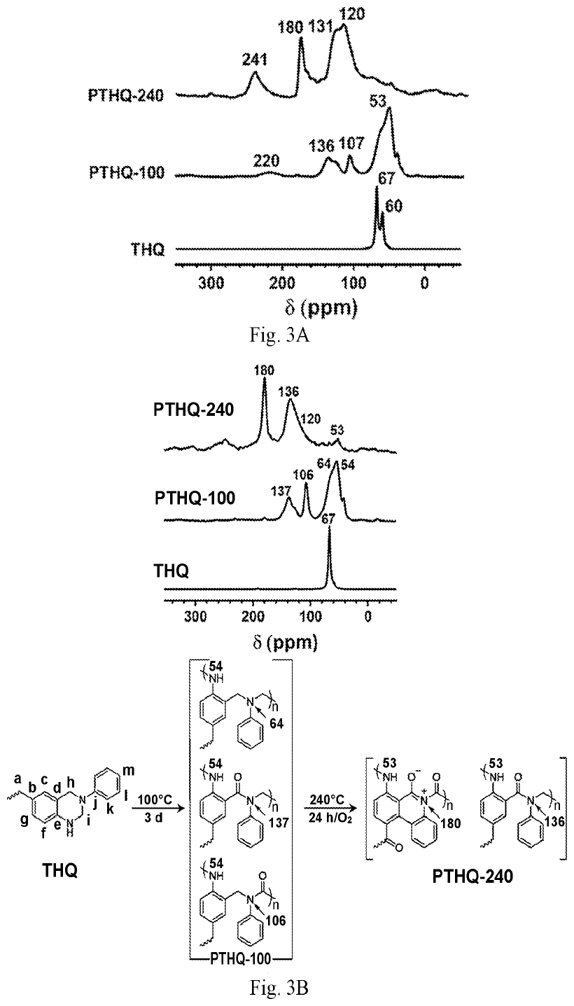

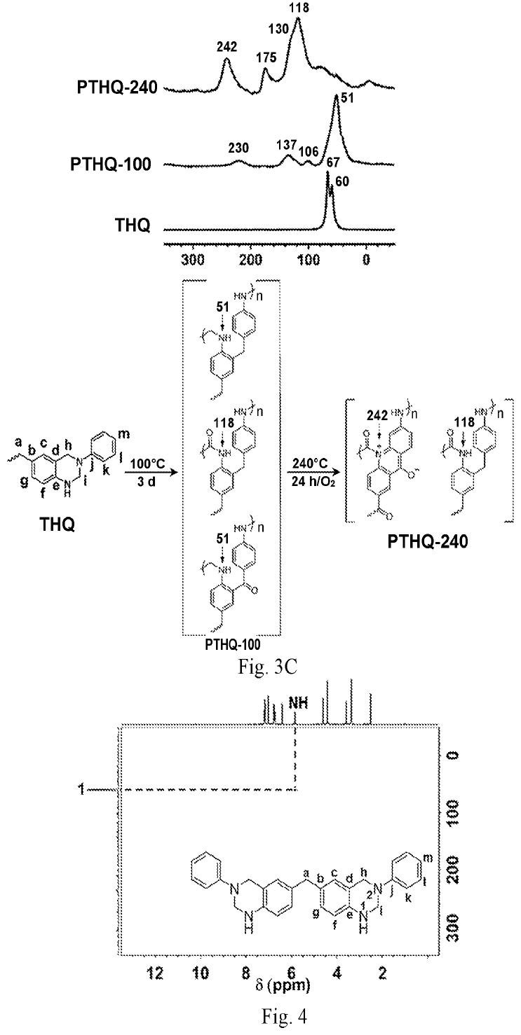

The SS ¹⁵N NMR spectra of both aniline and anthranilic enriched (FIG. 3A), aniline enriched (FIG. 3B), and anthranilic enriched (FIG. 3c) THQ monomers, PTHQ-100, and PTHQ-240 (each prepared according to the method described in Example 4) are shown in FIG. 3. Starting with the ¹⁵N-enriched THQ monomers, the peak at 60 ppm was assigned to the secondary N coming from anthranilic acid while the peak at 67 ppm was assigned to the tertiary N coming from aniline. The assignment of the peaks was based on the ¹H-¹⁵N HSQC NMR correlation experiment (see FIG. 4), which clearly identified the N to which the proton is attached. Both ¹⁵N-enriched THQ and secondary N enriched THQ showed peaks at 60 and 67 ppm while tertiary N enriched THQ showed only one peak at 67 ppm, which corresponds to the enriched N. The selective enrichment of tertiary N was responsible for masking the peak of secondary N at 60 ppm. In fact, the sharp peak in this case is not symmetric, and further NMR experiments with low contact time of 5 μs identified the peak of secondary N at 60 ppm (all other spectra were taken at contact time=3000 μs). It was observed that, in all three cases of THQ enrichment, the intensity of tertiary N dominated the NMR spectrum though it has no H attached to it (which helps in relaxation during the NMR experiment).

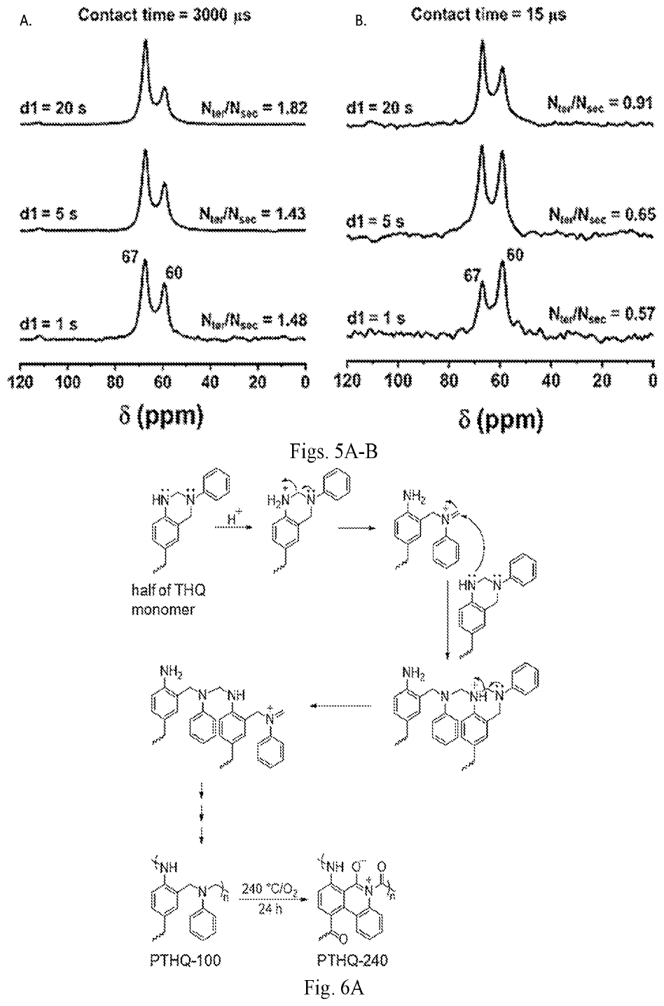

This observation conflicts with the basic NMR spectrometry principle that nuclei with H attached to them relax faster. This anomaly was further investigated by two sets of SS ¹⁵N NMR experiments on both ¹⁵N-enriched THQ samples as shown in FIG. 5. In the first set, the contact time was fixed at 3000 μs, and three different pulse delays (1, 5, and 20 s) were used to acquire the spectrum. Similar experiments were carried out in the second set by fixing the contact time at 5 μs. From this data, the area under the peak was calculated for secondary and tertiary N using Lorentzian fitting for each contact time, and In (intensity) was plotted against the pulse delay. The relaxation times (T1) for the secondary and tertiary N were obtained by the inverse of the above slope, which indicates that tertiary N relaxes faster than the secondary one (71 vs 91 s, respectively), confirming the higher peak intensity in the NMR spectrum.

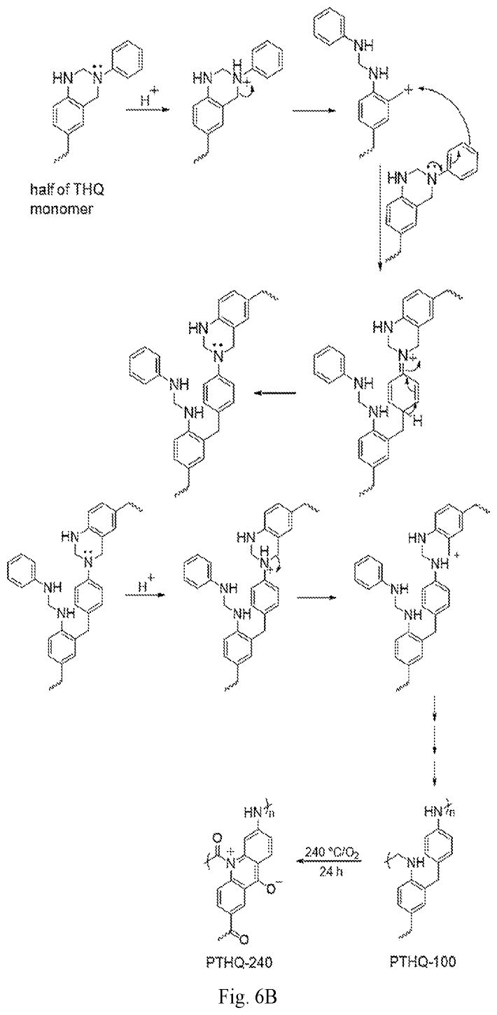

For ¹⁵N-enriched PTHQ-100 aerogels, the mechanism of ring opening polymerization is shown in FIG. 6A, and the corresponding repeating units of products formed after polymerization and ring fusion are shown in FIGS. 3B (aniline enriched PTHQ aerogels) and 3C (anthranilic enriched PTHQ aerogels). In an ideal case, only two peaks are expected after the ring opening polymerization in the range of 50-65 ppm, but, due to partial oxidation during the processing of gels, additional peaks with different functionalities were observed. The peak at 106 ppm was near the resonance of urea and the resonance at 137 ppm was assigned to amide functionality formed after the formation of carbonyl on carbons of Mannich bridge. Thus, the polymerization mechanism involves two processes: (a) acid catalyzed ring opening and (b) parallel formation of polyurea and polyamide due to partial oxidation on the Mannich bridges. The intensity of these two peaks (106 & 137 ppm) is higher in the case of aniline enriched PTHQ-100 aerogels as compared to both enriched and anthranilic enriched counterparts indicating that the tertiary N coming from the enriched aniline is responsible for the formation of urea and amide functionality in the polymeric backbone.

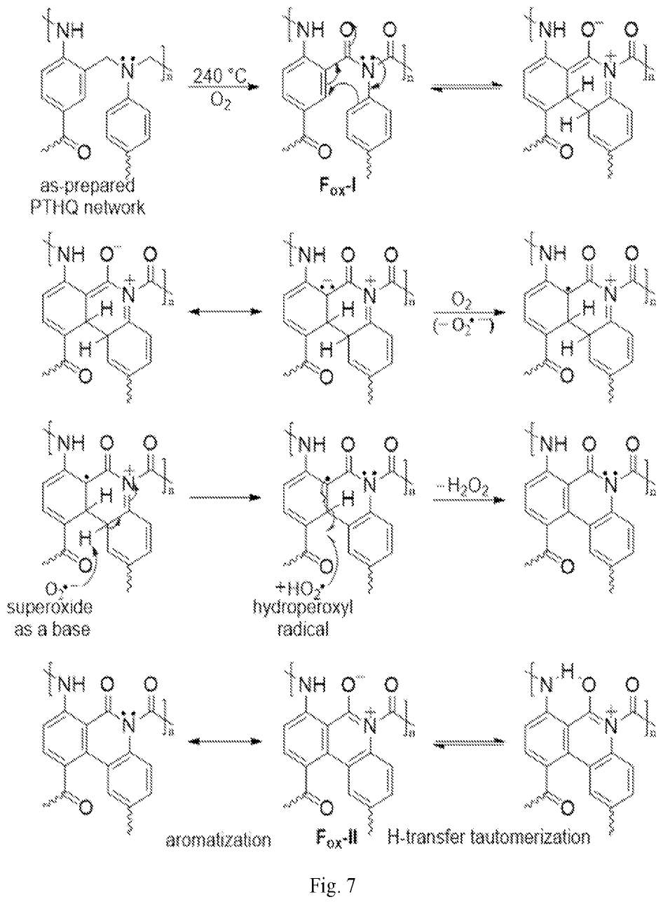

For ¹⁵N-enriched PTHQ-240 aerogels, no peaks in the aliphatic region were observed, indicating complete oxidation of the carbons of Mannich bridges. The peak at 137 ppm became intense in aniline enriched PTHQ signifying increase in amide functionality. The additional peak at 180 ppm was observed, which could be assigned to the tertiary N of the imide formed after complete oxidation. However, the oxidation does not stop after the formation of carbonyls. In fact, the carbonyls on the bridged carbons are formed by autooxidation in which a H-atom was abstracted by O₂ followed by addition of the resulting hydroperoxyl radical (HO₂.) at the benzylic positions. Subsequently, homolytic cleavage of O-OH bond results in the formation of carbonyl with release of H₂O. Further oxidation is driven by ring-fusion as shown in FIG. 7 and results in the formation of iminium ion. Thus, the final aniline enriched PTHQ-240 aerogels consist of an iminium functionality not an imide functionality appearing at 180 ppm. However, in the case of anthranilic enriched PTHQ-240, an additional peak at 242 ppm was observed, which was not present in aniline enriched PTHQ-240. In fact, aniline and anthranilic enriched PTHQ-240 show the same peak at 241 ppm. It is speculated that the N coming from anthranilic acid is involved in ring-fusion aromatization and is responsible for the peak at that position.

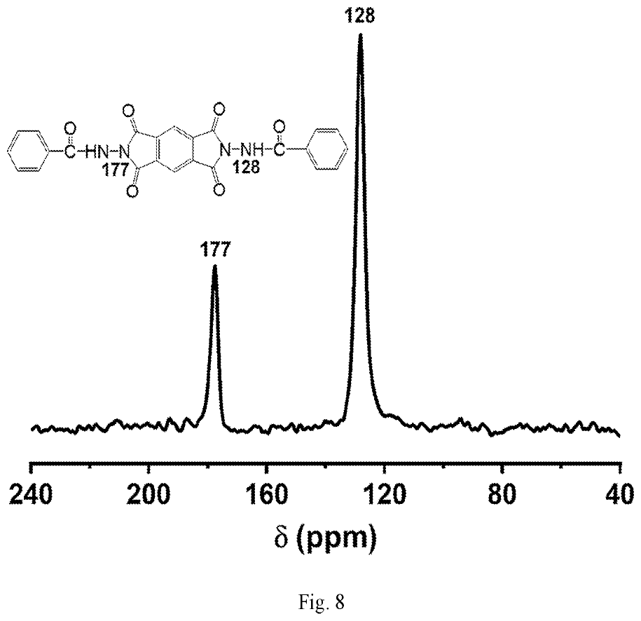

The mechanism of acid catalyzed ring opening has no possible route to form ring-fusion with secondary N of anthranilic acid. Thus, another mechanism of ring-opening polymerization was identified as shown in FIG. 6B, which goes via attack on the para position of aniline. The corresponding products formed after polymerization and aromatization are shown in FIG. 3C. Thus, the aromatized product in anthranilic enriched PTHQ-240 in which the secondary N is involved in ring-fusion follows a similar oxygen/superoxide/hydroperoxyl radical chemistry forming an iminium ion and is responsible for the ¹⁵N NMR resonance at 242 ppm (FIG. 3c). The ring-fusion aromatization and the product formed after aromatization in PTHQ-240 is supported by ab-initio Gaussian-09. Density Functional Theory (DFT) calculations using B3LYP data set, which showed that the aromatized structures are more stable than their corresponding precursors: by 73 kcal/mol for imide (mechanism A—FIG. 6A) and 48 kcal/mol for amide (mechanism B—FIG. 6B). The ratio of amide and aromatized product in PTHQ-240 was calculated from the integration of the corresponding peaks in the NMR. For that, the response factor was calculated using controls for amide and imide. The compound structure and the respective ¹⁵N NMR spectrum is shown in FIG. 8. The calculated amide:imide response factor is 1.00:0.3533. Thus, the amide:imide ratio in aniline enriched PTHQ-240 is 0.69 while for anthranilic enriched PTHQ-240 is 1.87.

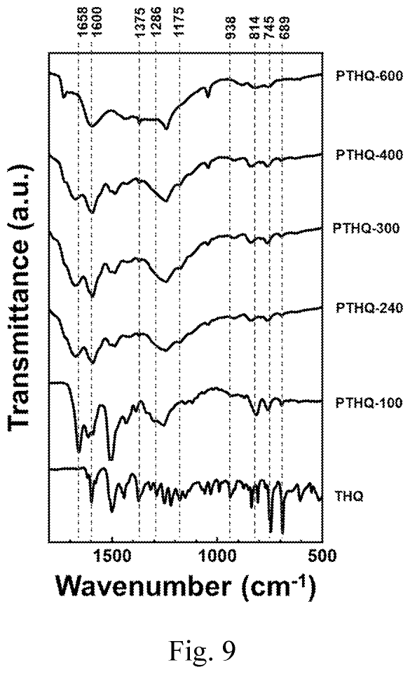

Further insight into the chemistry of polymerization and aromatization was obtained with FTIR. The FTIR spectra of THQ (prepared according to the methods described in Example 2), PTHQ-100 (prepared according to the methods described in Parts 2-3 of Example 3), and PTHQ-240 (prepared according to the methods described in Parts 2-4 of Example 3) up to 1800 cm⁻¹ are shown in FIG. 9. The strong absorptions of the THQ monomer at 745 cm⁻¹ and 689 cm⁻¹ correspond to out-of-plane (OOP) C—H bending vibrations from dangling aniline. After ring-opening, their intensity became extremely weak, signifying the involvement of the aniline moiety (I) in polymerization. After polymerization, an additional peak at 814 cm⁻¹ was observed in PTHQ-100 and was attributed to OOP C—H bending of a para substituted aromatic ring, confirming the mechanism B (FIG. 6B) of polymerization. The absorption at 1175 cm⁻¹ is attributed to Ar—C—Ar stretching between two aromatic rings, which survives the polymerization in accordance with ¹³C NMR but becomes weak due to partial oxidation. The absorption at 1286 cm⁻¹ and 1375 cm⁻¹ is attributed to C—N stretching and C—H bending, respectively. Also, a new absorption was observed in the range of 1658 cm⁻¹, which was assigned to C—O stretching of amide. Based on FTIR, PTHQ polymerization goes via additional para-coupling of aniline via ring opening into an iminium ion, which undergoes electrophilic aromatic substitution at the activated para-position of aniline, resulting in a tightly cross-linked polymer structure.

The formation of intermediates during polymerization and aromatization were tracked with CHNO analysis. The percent amount of oxygen was calculated via % O=100−(% C+% H+% N). The calculated CHNO weight percentages of plausible structures for PTHQ-100 and PTHQ-240 formed via Mechanism A are shown in Table 1.

TABLE 1
Calculated CHNO weight percent of plausible partially and fully oxidized PTHQ
forms with ring-fused structure (Mechanism A)

| Polymer | %C | %H | %N | %O |
|---|---|---|---|---|
| Experimental PTHQ-100 | 76.82 ± 0.10 | 6.23 ± 0.04 | 11.75 ± 0.06 | 5.29 ± 0.09 |
| 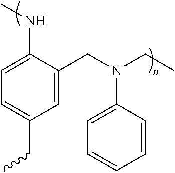 | 80.52 | 6.52 | 12.96 | — |
| Theoretical PTHQ | | | | |
| 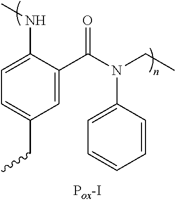 | 75.63 | 5.25 | 12.16 | 6.96 |
| 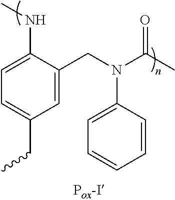 | 75.63 | 5.25 | 12.16 | 6.96 |
| Experimental PTHQ-240 | 66.63 ± 0.12 | 3.10 ± 0.25 | 11.83 ± 1.50 | 18.44 ± 1.13 |
| 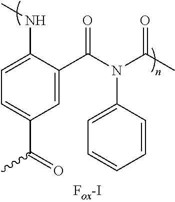 | 69.32 | 3.61 | 11.15 | 15.92 |
| 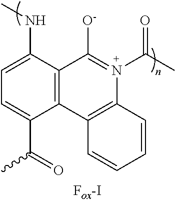 | 69.88 | 2.83 | 11.24 | 16.05 |
| 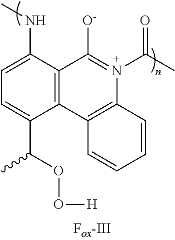 | 67.44 | 3.12 | 10.85 | 18.59 |

Furthermore, the calculated CHNO weight percentages of plausible structures for PTHQ-100 and PTHQ-240 formed via Mechanism B are shown in Table 2.

TABLE 2
Calculated CHNO weight percent of plausible partially and fully oxidized PTHQ
forms with ring-fused structure (Mechanism B)

| Polymer | % C | % H | % N | % O |
|---|---|---|---|---|
| Experimental PTHQ-100 | 76.82 ± 0.10 | 6.23 ± 0.04 | 11.75 ± 0.06 | 5.29 ± 0.09 |
| 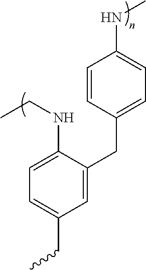 | 80.52 | 6.52 | 12.96 | — |
| Theoretical PTHQ | | | | |
| 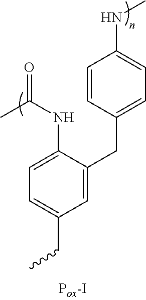 | 75.63 | 5.25 | 12.16 | 6.96 |
| 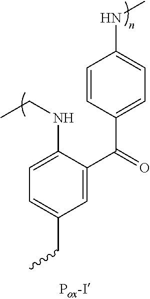 | 75.63 | 5.25 | 12.16 | 6.96 |
| Experimental PTHQ-240 | 66.63 ± 0.12 | 3.10 ± 0.25 | 11.83 ± 1.50 | 18.44 ± 1.13 |
| 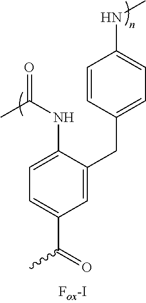 | 69.32 | 3.61 | 11.15 | 15.92 |
| 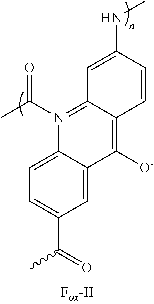 | 69.88 | 2.83 | 11.24 | 16.05 |
| 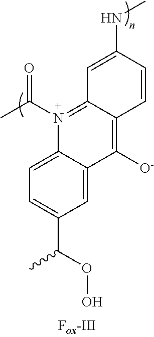 | 67.44 | 3.12 | 10.85 | 18.59 |

Calculated CHNO values of the intermediates are the same for Mechanism A and Mechanism B as both consist of the same number and types of atoms. Turning to Table 1, for the idealized PTHQ-100structure shown in FIG. 3B, the calculated values of CHNO differ significantly from the experimental values. This variation is due to partial oxidation on the Mannich bridge during polymerization. Pₒₓ-I and Pₒₓ-I′ are the two possible partially oxidized structures for PTHQ-100, of which are responsible for the formation of urea and amide functionality respectively in the polymeric backbone. Considering all three structures together (idealized PTHQ-100, Pₒₓ-I, and Pₒₓ-I′), the calculated CHNO values are closer to the experimental values, implying that the polymeric backbone of PTHQ-100 is a combination of these partially oxidized products. Moving on to PTHQ-240, the fully oxidized structure Fₒₓ-I undergoes ring fusion aromatization to form Fₒₓ-II as shown in FIG. 7. However, the experimental CHNO values for PTHQ-240 vary from the calculated values mostly in terms of carbon and oxygen. As explained earlier, the introduction of carbonyls on the-CH₂-bridges goes first via formation of peroxide. It was speculated that there is a presence of some repeating units with peroxide in the PTHQ-240 polymeric backbone. The peroxide containing structure is represented by Fₒₓ-III, and the respective calculated CHNO values are in close agreement with the experimental values for PTHQ-240.

### 3. Chemical Characterization of PTHQ-300, PTHQ-400, PTHQ-600, C-PTHQ-800, and EC-PTHQ-1000 Aerogels.

The aerogels formed after aromatization were used to further investigate the pyrolysis process. For this purpose, PTHQ-240 aerogels were pyrolyzed at three different temperatures (300° C., 400° C., and 600° C.) under flowing high-purity argon as described in Part 5 of Example 3. In addition, terminal pyrolysis was carried out at 800° C. as described in Part 5 of Example 3, and these aerogels are referred to as C-PTHQ-800. Fresh aerogel samples were used at every pyrolysis temperature. Chemical transformations along pyrolysis were monitored by ¹³C solid-state NMR and FTIR while the chemical composition was monitored by elemental analysis. XPS was used specifically to characterize heteroatoms in the C-PTHQ-800 aerogels.

The ¹³C solid state NMR spectra of PTHQ-300, PTHQ-400, and PTHQ-600 aerogels are shown in FIG. 2. There is no drastic variation in the pyrolysis products at different temperatures as compared to PTHQ-240, suggesting that oxidative aromatization is responsible for accelerated chemical changes that otherwise are observed during conventional pyrolytic carbonization at higher temperatures under inert atmosphere. Similar observations were made from FTIR spectra of PTHQ-300, PTHQ-400, and PTHQ-600 (FIG. 9), showing two major absorptions at around 1600 cm⁻¹ and 1660 cm⁻¹ surviving pyrolysis.

The elemental analysis data along the pyrolysis is shown in Table 3.

TABLE 3
Representative elemental analysis data for PTHQ
aerogels pyrolyzed at different temperatures

| Sample | % C | % H | % N | % O |
|---|---|---|---|---|
| PTHQ-100 | 76.92 | 6.20 | 11.69 | 5.91 |
| PTHQ-240 | 66.62 | 2.68 | 10.65 | 18.24 |
| PTHQ-300 | 67.51 | 3.10 | 10.93 | 18.45 |
| PTHQ-400 | 70.69 | 2.96 | 11.28 | 15.06 |
| PTHQ-600 | 79.83 | 2.30 | 10.04 | 7.83 |
| C-PTHQ-800 | 84.29 | 1.01 | 7.13 | 7.57 |
| EC-PTHQ-1000 | 85.72 | 1.76 | 3.62 | 9.20 |

All the samples were analyzed three times. Slight increase in the C content and decrease in N content was observed for PTHQ-300 as compared to PTHQ-240, indicating a start of carbonatization process. Along further pyrolysis, the system becomes richer in C and deficient in N with steady decrease in H content. At 800° C., PTHQ aerogels have produced carbons with chemical composition: C: 84.29 w/w, H: 1.01 w/w, N: 7.13 w/w, O: 7.57 w/w.

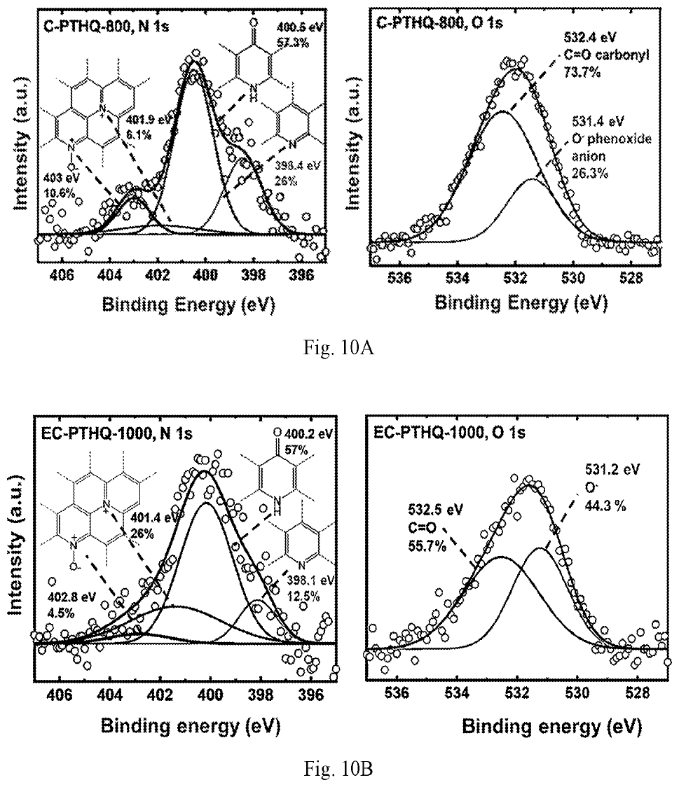

The high resolution XPS spectra of O 1 s and N 1 s for C-PTHQ-800 and for EC-PTHQ-1000 are shown in FIGS. 10A and 10B, respectively. Spectra were fitted with two Gaussians for O and four Gaussians for N. As shown in FIG. 10A, the O Is spectra for C-PTHQ-800 show two absorptions: one at 532.4 eV, which indicates the presence of carbonyl oxygen (73.7%) and one at 531.4 eV, which corresponds to phenoxide O⁻ (26.3%). XPS spectra of N 1 s for C-PTHQ-800 were centered at 398.4 eV (pyridinic N, 26.0%), at 400.5 eV (pyridonic N, 57.3%), at 401.9 eV (quaternary N, 6.1%), and at 403.0 eV (pyridine oxide, 10.6%).

Thus, oxidation of PTHQ aerogels gave well defined products (PTHQ-240) with extensive ring-fusion aromatization along their backbone, of which survived for several higher temperatures afterwards. This early insertion of oxidation step in the carbonization process is important to get sturdy carbons, which are difficult to be obtained without ring fusion.

### Example 7

### Physical and Structural Characterization and Porosity Studies of PTHQ-100, PTHQ-240, PTHQ-300, PTHQ-400, PTHQ-600, C-PTHQ-800, and EC-PTHQ-1000 Aerogels

### 1. Physical Characterization Methods

Bulk densities (b) were calculated from the weight and physical dimensions of the PTHQ-100, PTHQ-240, PTHQ-300, PTHQ-400, PTHQ-600, C-PTHQ-800, and EC-PTHQ-1000 aerogel samples prepared according to the methods described in Example 3. Skeletal densities (ps) were measured using helium pycnometry with a Micromeritics AccuPyc II 1340 instrument.

### 2. Structural Characterization Methods

Scanning electron microscopy (SEM) was conducted on Au-coated PTHQ-100, PTHQ-240, C-PTHQ-800, and EC-PTHQ-1000 aerogel samples prepared according to the methods described in Example 3 with a Hitachi Model S-4700 field-emission microscope.

In addition, the fundamental building blocks of PTHQ-100, PTHQ-240, C-PTHQ-800, and EC-PTHQ-1000 aerogels were probed with small-angle X-ray scattering (SAXS) using approximately 2 mm thick disks cut with a diamond saw. SAXS was conducted with a PANalytical X'Pert Pro Multipurpose Diffractometer (MPD) configured for SAXS using Cu Kα radiation (wavelength=1.54 Å), a 1/32° SAXS slit, a 1/16° anti-scatter slit on the incident beam side, and a 0.1 mm anti-scatter slit together with a Ni 0.125 mm automatic beam attenuator on the diffracted beam side. PTHQ-100, PTHQ-240, C-PTHQ-800, and EC-PTHQ-1000 aerogel samples were placed in circular holders between thin Mylar sheets, and scattering intensities were measured by running 20 scans from −0.1° to 5° with a point detector in the transmission geometry. All scattering data were reported in arbitrary units of scattering intensity as a function of Q, which represents the momentum transferred during a scattering event. Scattering data were fitted to the Beaucage Unified Model and applied with the Irena SAS tool for modeling and analysis of small-angle scattering within the Igor Pro application (a commercial scientific graphing, image processing, and data analysis software from Wave Metrics, Portland, OR).

### 3. Porosity Study Methods

The pore structure of PTHQ-100, PTHQ-240, C-PTHQ-800, and EC-PTHQ-1000 aerogel samples were probed with N₂-sorption porosimetry at 77 K using either Micromeritics ASAP 2020 or a TriStar II 3020 version 3.02 surface area and porosity analyzer. Before the analysis, the aerogel samples were degassed for 24 hours under vacuum at 80° C. Total surface areas, σ, were determined via Brunauer-Emmett-Teller (BET) method from the N₂-sorption isotherms. Micropore surface areas were calculated via t-plot analysis of the N₂-sorption isotherms using the Harkins and Jura model. Average diameters of pores above 2 nm in size were calculated using the 4×V/σ method, where the specific pore volume, V, was set equal either to V_{Total}=(1/ρ_{b})−(1/ρₛ) or to Vₘₐₓ, which is the single highest volume of N₂ adsorbed along the N₂-sorption isotherm as P/P₀→1. Micropore analysis was conducted either with low-pressure N₂-sorption at 77 K using a Micromeritics ASAP 2020 instrument equipped with a low-pressure transducer or with CO₂ adsorption up to 760 Torr (relative pressure P/P₀=0.03) at 273 K using the Micromeritics TriStar II 3020 system mentioned above.

Importantly, prior to low-pressure N₂-sorption analysis at 77 K, a third degassing step was carried out under 1 μm Hg at 120° C. directly on the analysis port of the Micromeritics ASAP 2020 instrument. Micropore volumes were determined either from the low-pressure N₂-sorption data at 77 K or from the CO₂ adsorption data at 273 K by using the Dubinin-Radushkevich (DR) equation. Average micropore sizes were calculated using the 4×V/micropore area method, where the specific micropore volume, V, was set equal to the micropore volumes calculated via the DR model either from the low-pressure N₂ or the CO₂ adsorption data. The micropore area was set equal to the micropore surface area obtained from the t-plot method. Micropore size distributions for pores <1 nm were obtained from CO₂ adsorption data using density function theory (DFT) method.

### 4. Macroscopic Properties of PTHQ-100 and PTHQ-240 Aerogels

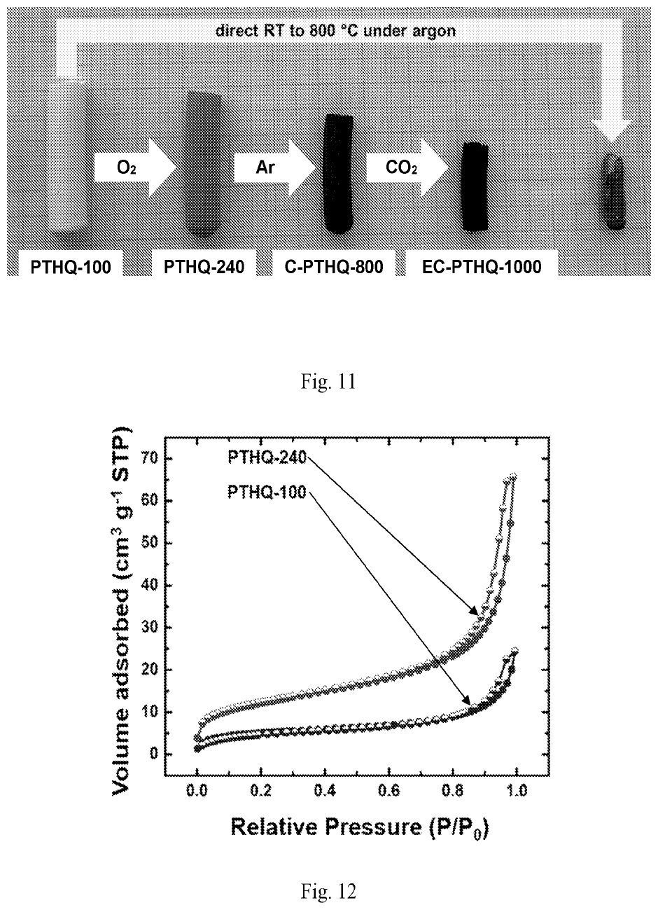

FIG. 11 shows PTHQ-100 (prepared according to methods described in Part 3 of Example 3) and PTHQ-240 (prepared according to methods described in Part 4 of Example 3) aerogel monoliths.

Notably, PTHQ-100 aerogels shrunk significantly relative to their molds. Linear shrinkage is around 25% for PTHQ-100 aerogels. Most of the shrinkage occurred during aging, and no significant shrinkage was observed during solvent exchange from DMF to acetone or during drying. Oxidation under O₂ caused an additional shrinkage of 18% relative to the mold for PTHQ-240 as shown in Table 4.

TABLE 4
Material properties of PTHQ aerogels

| | Linear | | Skeletal | Porosity | Specific pore volume | |
|---|---|---|---|---|---|---|
| | shrinkage | Bulk density, | density, | II [% | [cm³ g⁻¹] | |
| Sample | [%]^{a, b)} | ρ_{b} [g cm⁻³]^{a)} | ρₛ [g cm⁻³]^{c)} | v/v]^{d)} | V_{Total}^{e)} | V_{1.7-300}_{_{—}}ₙₘ^{f)} |
| PTHQ-100 | 25.₁ ± 2 | 0.195 ± 0.008 | 1.575 ± 0.003 | 88 | 4.50 | 0.03 |
| PTHQ-240 | 38.83 ± 0.05 | 0.236 ± 0.005 | 1.442 ± 0.009 | 83 | 3.54 | 0.03 |
| C-PTHQ-800 | 70.₈ ± 1 | 0.225 ± 0.001 | 2.19₇ ± 0.018 | 90 | 3.46 | 0.09 |
| EC-PTHQ- | 85.60 ± 0.03 | 0.15₂ ± 0.02 | 1.84₃ ± 0.012 | 92 | 6.12 | 0.21 |
| 1000 | | | | | | |
| PTHQ-300 | 28.6 ± 0.3 | 0.258 ± 0.005 | 1.36₉ ± 0.013 | 81 | 3.14 | 0.04 |
| PTHQ-400 | 29.3 ± 0.6 | 0.231 ± 0.002 | 1.394 ± 0.007 | 83 | 3.60 | 0.05 |
| PTHQ-600 | 34.4 ± 0.4 | 0.216 ± 0.005 | 1.54₆ ± 0.012 | 86 | 3.98 | 0.06 |

| | | | | Average pore diam. | |
|---|---|---|---|---|---|
| | Specific pore volume | Surface area [m² g⁻¹] | | [nm] via 4 × V/σⁱ⁾ | |
| | [cm³ g⁻¹] | BET, | | V = | V = |
| Sample | Vₘₐₓ^{g)} | σ | Micropore^{h)} | V_{Total} | Vₘₐₓ |
| PTHQ-100 | 0.03 | 18 | 3 | 1136 | 7.9 |
| PTHQ-240 | 0.10 | 42 | 4 | 331 | 9.6 |
| C-PTHQ-800 | 0.26 | 346 | 263 | 40 | 3.0 |
| EC-PTHQ- | 1.02 | 1680 | 1050 | 15 | 2.4 |
| 1000 | | | | | |
| PTHQ-300 | 0.05 | 23 | 3 | 547 | 8.7 |
| PTHQ-400 | 0.07 | 37 | 8 | 385 | 7.6 |
| PTHQ-600 | 0.27 | 432 | 342 | 37 | 2.5 |

^{a)}Average of five samples;
^{b)}All values relative to molds;
^{c)}Single sample, average of 50 measurements;
^{d)}Via II = 100 × (ρₛ − ρ_{b})/ρₛ;
^{e)}V_{Total} = (1/ρ_{b}) − (1/ρₛ);
^{f)}BJH-desorption cumulative pore volume;
^{g)}Vₘₐₓ: single point N₂ adsorption at 77 K as P/P₀→ 1.0
^{h)}Via the t-plot method;
ⁱ⁾For V_{Total} and Vₘₐₓ refer to footnotes e and f.

This excessive shrinkage could be attributed to molecular rigidity achieved due to a higher degree of crosslinking during the aromatization. The bulk density (ρ_{b}) of PTHQ-100 is 0.195±0.008 g cm⁻³ and is increased to 0.236±0.005 g cm⁻³ for the PTHQ-240 sample. The skeletal density (ρₛ) for PTHQ-100 is 1.575±0.003 g cm⁻³ and is decreased to 1.442±0.009 g cm⁻³ for PTHQ-240 after oxidation. The open porosity (II), calculated as percentage of empty space via Π=100×(ρₛ−ρₕ)/ρₛ, varied from 88% v/v (PTHQ-100) to 83% v/v (PTHQ-240).

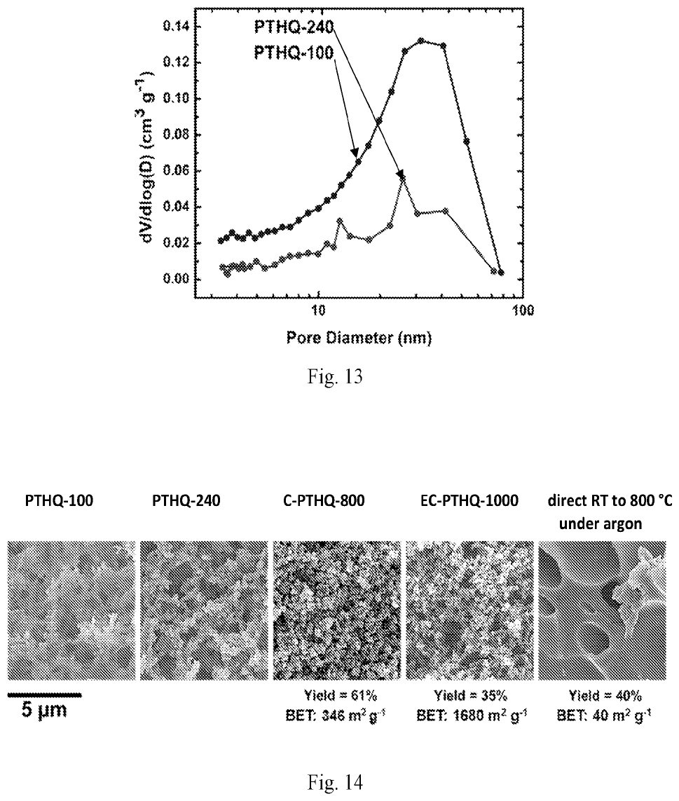

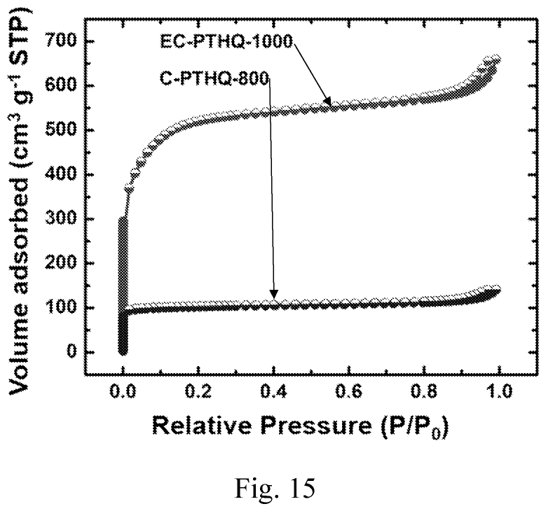

In addition, quantitative evaluation of the porous structure for PTHQ-100 and PTHQ-240 (prepared according to methods described in Parts 3 and 4 of Example 3, respectively) was investigated with N₂-soprtion, and this data are shown in Table 4. As shown in FIG. 12, the N₂-sorption isotherms show no sign of microporosity. Both isotherms show narrow hysteresis loops without saturation plateau, indicating mostly macroporous materials. Specific pore volume, in the range of 1.7-300 nm, was calculated with the Barrett, Joyner, and Halenda (BJH) method (FIG. 13) and found to be negligible for both samples as compared to the total specific pore volume calculated via V_{Total}=(1/ρ_{b})−(1/ρₛ). V_{Total} accounts for the entire porosity of both PTHQ-100 and PTHQ-240. Average pore diameter, calculated via V_{Total}/σ method, was 1136 nm for PTHQ-100. After oxidation, the average pore diameter decreased dramatically to 331 nm due to molecular contraction during oxidative aromatization. The BET surface area, σ, is low for both samples: σ=18 and 42 m² g⁻¹ in PTHQ-100 and PTHQ-240, respectively. However, 10-15% of the BET surface area was attributed to micropores (calculated with the Harkins and Jura model).

### 5. Microscopic Properties of PTHQ-100 and PTHQ-240 Aerogels

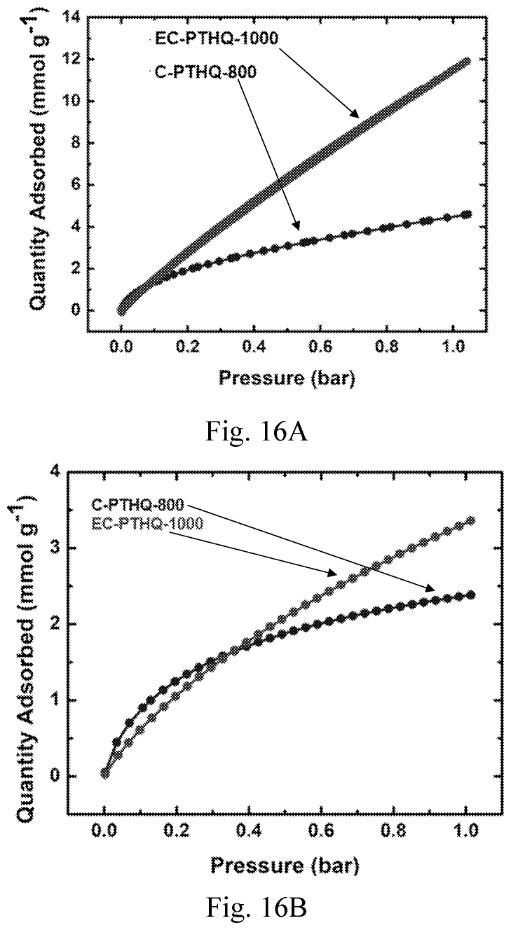

Skeletal framework of PTHQ-100 and PTHQ-240 samples (prepared according to methods described in Part 1 of this Example) was probed with scanning electron microscopy (SEM) and small-angle X-ray scattering (SAXS). According to SEM (FIG. 14), the skeletal framework of PTHQ-100 shows random dendritic growth of the particles. After oxidation, they form nanoparticles of nearly similar size. Particles aggregate to form large clusters during oxidation.

The presence of large voids in SEM are consistent with the macroporous character of the sample as probed by N₂-sorption. SAXS was used for the quantitative evaluation of the skeletal framework, and the data analyzed with the Baucage Unified Model. Scattering plots for PTHQ-100 and PTHQ-240 could be fitted in two regions. The primary particle radii obtained from SAXS were 104.67±1.85 nm and 102.78±3.87 nm for PTHQ-100 and 240, respectively. The primary particle diameter obtained from N₂-sorption via r=3/ρₛσ, (106 nm) show remarkable agreement with the value calculated from SAXS for the PTHQ-100 sample. However, for the PTHQ-240 sample, the particle size calculated by SAXS and N₂-sorption diverge (103 nm versus 49 nm, respectively).

### 6. Macroscopic Properties of PTHQ-300, PTHQ-400, PTHQ-600, C-PTHQ-800, and EC-PTHQ-1000 Aerogels

The material properties for PTHQ-300, PTHQ-400, PTHQ-600, C-PTHQ-800, and EC-PTHQ-1000 aerogels are shown in Table 4. Notably, along different pyrolysis temperatures, bulk density (ρₕ) and skeletal density (ρₛ) vary randomly. Specifically, ρₛ of C-PTHQ-800 (2.197 g cm⁻¹) is close to the range of expected densities for amorphous carbon (1.8-2.0 g cm⁻¹) while, for EC-PTHQ-1000, ρₛ is (1.843 g cm⁻¹).

In terms of porosity, no significant variation was observed relative to parent carbons. The porosity of all pyrolysis products between 300-600° C. remains high (>80%). For C-PTHQ-800 and EC-PTHQ-1000, the porosity is in the range of 90-92%. Along pyrolysis, there is a small amount of additional shrinkage. After carbonization, C-PTHQ-800 shrunk 45% relative to their parent aerogel PTHQ-100 while EC-PTHQ-1000 shrunk to 60%. Overall, total shrinkage is 71% and 85%, respectively, for carbonized and etched samples relative to the molds. The carbonization yield was found to be 61%, and the yield of reactive etching was 35%.

Finally, meso-and macroporosity was probed with medium pressure N₂-adsorption at 77 K for C-PTHQ-800 and EC-PTHQ-1000.

### 7. Microscopic Properties of C-PTHQ-800 and EC-PTHQ-1000 Aerogels

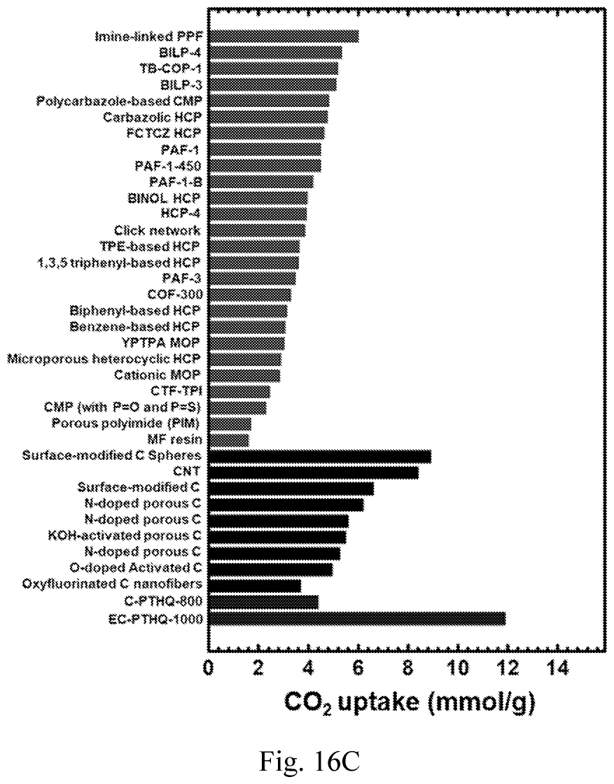

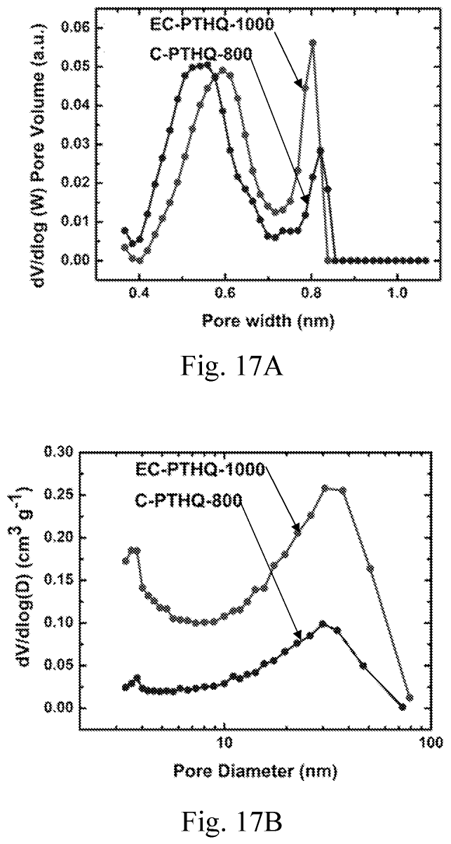

Particle size data for the C-PTHQ-800 and EC-PTHQ-1000 samples were obtained from SAXS measurements. Microporosity was evaluated mainly for C-PTHQ-800 and EC-PTHQ-1000 by low pressure N₂-adsoprtion (FIG. 15) at 77 K and CO₂ adsorption at 273 K (FIG. 16A), and this data summarized in Table 4.

As shown in FIG. 14, the skeletal framework of C-PTHQ-800 and EC-PTHQ-1000 was evaluated by SEM, which showed that these aerogels consist of loose assemblies of nanoparticles forming meso-and macropores. In comparison with PTHQ-100, pyrolysis resulted in the fusion of particles, which is evident in higher-density and was observed for C-PTHQ-800 and EC-PTHQ-1000. The etching process introduced wider macropores and micropores. However, the ring-fusion aromatization at 240° C. played a huge role in obtaining sturdy monolithic carbon and etched carbon PTHQ aerogels with well-defined skeletal framework. The carbonization of PTHQ-100 without aromatization (SEM image shown in FIG. 14) results in complete loss of skeletal framework with 20% less carbonization yield than carbonization after treatment at 240° C. The SAXS data for C-PTHQ-800 and EC-PTHQ-1000 samples could be fitted in four regions. The secondary particle radii obtained from SAXS data for C-PTHQ-800 and EC-PTHQ-1000 was R₂=100.09±3.24 nm and 99.37±3.01 nm, respectively. The primary particle radii obtained from SAXS are higher than the radii probed by N₂-sorption: 15 nm vs 22 nm for C-PTHQ-800 and 11 nm vs 7 nm for EC-PTHQ-1000. This difference could be due to limitation of SAXS in detecting primary particles underneath the polymer layer.

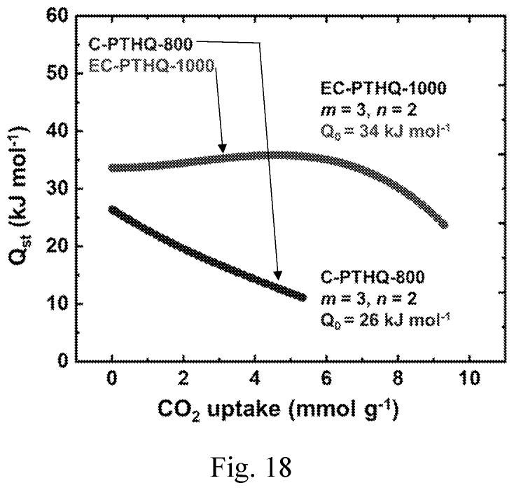

The N₂-sorption isotherms for C-PTHQ-800 and EC-PTHQ-1000 carbon aerogels are shown in FIG. 15. They were Type I with narrow saturation plateau and hysteresis loop signifying micro-and macroporosity. The comparison of carbonized and etched aerogels with their parent aerogel PTHQ-100 show remarkable difference in N₂-sorption (FIG. 12) with parent aerogels having much less adsorption. At low relative pressure, both C-PTHQ-800 and EC-PTHQ-1000 aerogels show a rapid increase of the volume of N₂ adsorbed at P/P₀<<0.1, indicating microporosity. This increase in microporosity was observed for temperatures >400° C. The microporosity was confirmed by pore size distribution analysis using the DFT method (FIG. 17A). The EC-PTHQ-1000 sample show dramatic increase in the micropore volume after etching and higher N₂-sorption than C-PTHQ-800. The adsorption increased more than 6-fold after etching. Thus, reactive etching opened access to the closed pores of C-PTHQ-800 and made existing micropores slightly wider. The evaluation of open porosity was conducted by comparing total pore volumes calculated via V_{Total}=(1/ρₕ)−(1/ρₛ) with Vₘₐₓ (the total volume of N₂ uptaken during N₂-sorption porosimetry as P/P₀→1). The data for both the samples are shown in Table 4. Decrease in average pore diameter was observed along the pyrolysis, which, after etching, was reduced dramatically.

In addition, BET surface area, σ, was calculated from medium pressure N₂-sorption, followed by t-plot analysis of micropore area with the Harkins and Jura method. The surface area of C-PTHQ-800 aerogel is 346 m² g⁻¹. There is no significant variation in the surface area with pyrolysis temperature until 400° C. The surface area increased dramatically after complete carbonization—and especially after etching—reaching value of 1680 m² g⁻¹ for EC-PTHQ-1000. BET surface area attributed to micropores was 76% and 42% for C-PTHQ-800 and EC-PTHQ-1000 samples, respectively. Overall, carbonization leaves the mesopore surface area intact, creating new surface area within micropores while etching significantly increased the micropore surface area.

Pore size distribution of C-PTHQ-800 and EC-PTHQ-1000 aerogels are shown in FIG. 17B. The meso and micro pore size distribution for both the aerogels are identical to one another. However, etching process resulted in wider cavities, slightly shifting the size distribution towards larger sizes. The DFT pore volume (<1 nm) contributes up to 36% for C-PTHQ-800 and 15% for EC-PTHQ-1000, indicating that samples consist of larger micropores not accounted for by DFT method.

### Example 8

### Gas Sorption Studies of C-PTHQ-800 and EC-PTHQ-1000 Aerogels

### 1. Method

The CO₂ adsorption capacity of the C-PTHQ-800 and EC-PTHQ-1000 aerogels at 273 K and 298 K was determined using a Micromeritics TriStar 3020 instrument. C-PTHQ-800 and EC-PTHQ-1000 aerogel samples (prepared according to the method described in Parts 5-6 of Example 3) were placed in analysis tubes (provided by Micrometrics) and degassed overnight (i.e., 18 hours) at 120° C. The weight of each degassed sample was determined from the difference between the weight of tube and the sample in the tube.

To begin the analysis, the tubes were placed on a TriStar analysis port in the instrument. An ice water bath was used to maintain the samples at 273 K, and a normal room temperature water bath was used to maintain the samples at 298 K. Once the samples reached the correct temperature, the tubes were then purged with CO₂ gas. Then, the pressure was adjusted from 0 to 1 bar, and the CO₂ adsorption capacity of each sample was recorded and determined as the pressure increased. The complete analysis (including time taken between each measurement) took 6 to 8 hours.

FIGS. 16A-B show the CO₂ adsorption isotherms at 273 K and 298 K for the C-PTHQ-800 and EC-PTHQ-1000 aerogel samples.

### 2. Calculation of the Isosteric Heats of CO₂ Adsorption

The adsorbate-adsorbent interactions and characterization of the energetic heterogeneity of the surface of the C-PTHQ-800 and EC-PTHQ-1000 aerogels was studied and quantified by calculating the isosteric heats of CO₂ adsorption for the aerogels. The isosteric heats of CO₂ adsorption were calculated using the Virial method. For this, the CO₂ adsorption isotherms at 273 K (FIG. 16A) and 298 K (FIG. 16B) were fitted simultaneously with a Virial-type Equation (1) using the OriginPro 9.7 software package:

$$ln P = ln N + \frac{1}{T} {\sum }_{i = 0}^{m} {a}_{i} {N}^{i} + {\sum }_{i = 0}^{n} {b}_{i} {N}^{i} \qquad ( 1 )$$

The variables of Equation (1) represent the following: P is pressure in Torr, N is the adsorbed amount in mmol g⁻¹, T is the absolute temperature, aᵢ and bᵢ are the Virial coefficients, and m and n are the number of coefficients needed in order to fit the isotherms adequately. Using the least squares method, the values of m and n were gradually increased until the sum of the squared deviations of the experimental points from the fitted isotherm was minimized. Data were fitted well with m=4 and n=2 for EC-PTHQ-1000 and m=3 and n=2 for C-PTHQ-800.

The values of do to am were introduced into Equation (2), and the isosteric heats of adsorption (Qₛt) were calculated as a function of the surface coverage (N).

$${Q}_{st} = - R {\sum }_{i = 0}^{m} {a}_{i} {N}^{i} \qquad ( 2 )$$

The variables of Equation (2) represent the following: R is the gas constant (8.314 J mol⁻¹ K⁻¹) and Qₛₜ is given in kJ mol⁻¹. The common term in Equation (2) for all N, Q₀, corresponds to i=0 and is given by Equation (3).

$${Q}_{0} = - {Ra}_{0} \qquad ( 3 )$$

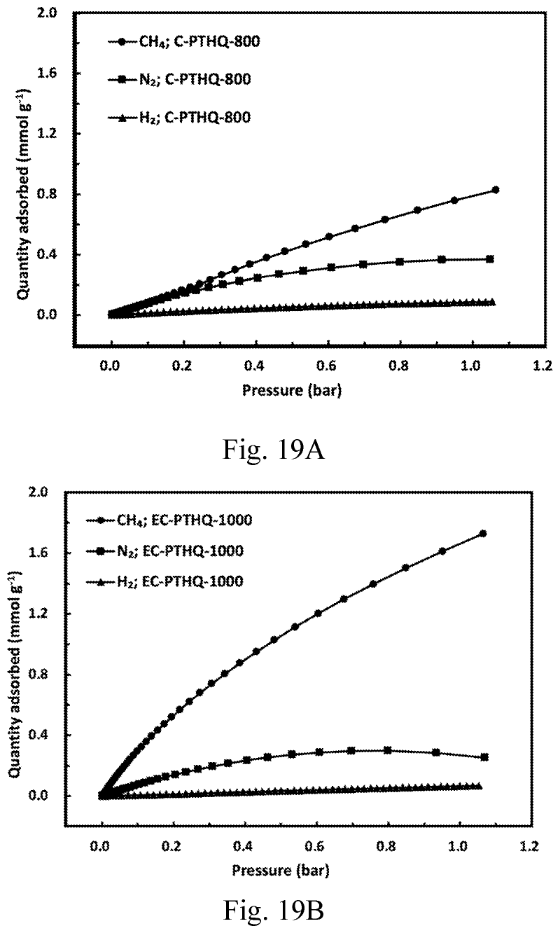

Q₀ is the heat of adsorption as coverage goes to zero and is a sensitive evaluator of the affinity of the adsorbate for the surface (i.e., the energy of interaction of CO₂ with the surface of the adsorbent). This data is shown in FIG. 18.

TABLE 5
Micropore analysis and CO₂ uptake at 0° C. of PTHQ carbon and etched carbon aerogels.

| | | | | | | | CO₂ uptake [mmol g⁻¹] at 273 K, 1 bar | | | |
|---|---|---|---|---|---|---|---|---|---|---|
| | | | | Micropore diameter [nm] via | | | | calculated from | | |
| | Specific micropore volume | | | 4 × V/(micropore area)^{d} | | | | | | specific micropore |
| | [cm³g⁻¹] | | | V from | V from | | measured | surface areas | | volumes |
| sample | DR(N₂) ^{a} | DR(CO₂)^{b} | DFT(CO₂) ^{c} | DR(N₂) | DR(CO₂) | average^{e} | experimentally | BET^{f} | Micropore ^{g} | DR(N₂)^{h} |
| C-PTHQ-800 | 0.16 | 0.36 ± 0.09 | 0.10 ± 0.01 | 2.50 | 4.67 | 3.58 | 4.41 ± 0.10 | 3.02 | 2.48 | 3.71 |
| EC-PTHQ- | 0.98 | 0.85 ± 0.05 | 0.14 ± 0.02 | 5.60 | 3.23 | 4.41 | 11.20 ± 0.9 | 16.11 | 10.53 | 22.66 |
| 1000 | | | | | | | | | | |

^{a} Via the Dubinin-Radushkevich (DR) method from N₂-sorption data obtained at 77 K using a low-pressure transducer (P/Pₒ ≤ 0.01). Single experiment;
^{b}Via the DR method from CO₂ adsorption data at 273 K up to relative pressure of 0.015;
^{c} Using the same data as in footnote (b) and applying the DFT method. Pore volumes correspond to pores <1 nm in size;
^{d}Calculated as indicated using the micropore surfaces areas obtained from N₂-sorption data via the t-plot method;
^{e}Calculated via 4 × V/(micropore area), where V = [V_{DR(N2)} + V_{DR(CO2)}]/2;
^{f}Calculated by dividing the BET surface area over the CO₂ cross sectional area (0.17 nm²), over the Avogadro's number;
^{g} Calculated by dividing the micropore surface area obtained from N₂-sorption data via the t-plot method over the CO₂ cross sectional area (0.17 nm²), over Avogadro's number;
^{h, i}Calculated by assuming that micropore volumes (via the DR(N₂) and the DFT(CO₂) methods - see footnotes a and c, respectively) are filled with liquid CO₂ (the density of liquid CO₂ at 273 K, was taken equal to the density of adsorbed CO₂ (1.023 g cm⁻³). Errors were calculated by applying rules of propagation of error.

### 3. Calculation of Adsorption Selectivities

Adsorption selectivities for one gas versus another were calculated as the ratios of the respective Henry's constants, K_{H}. The latter were calculated via another Virial model, whereas the single-component adsorption isotherms for each gas at 273 K were fitted according to Equation (4).

$$ln N = ln P + {K}_{1} + {K}_{2} N + {K}_{3} {N}^{2} + … = ln P + {\sum }_{i = 1}^{m} {K}_{i} {N}^{i - 1} \qquad ( 4 )$$

Fitting was carried out using the least squares method by varying the number of terms, until a suitable number of terms, m, described the isotherms adequately. Coefficients K₁, K₂, . . . Kₘ are characteristic constants for a given gas-solid system and temperature. The Henry's constant for each gas, K_{H}, is the limiting value of N/P as P→0 and is given by Equation (5).

$${K}_{H} = {lim}_{P \to 0} ( \frac{N}{P} ) ≅ {e}^{{K}_{1}} \qquad ( 5 )$$

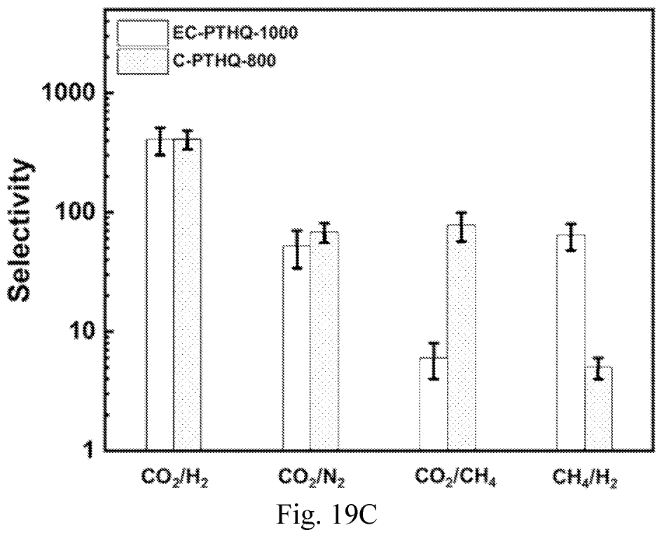

To calculate standard deviations, all isotherms obtained experimentally for each component were fitted individually. The K_{H} values from all isotherms were averaged, and the average values were used to calculate selectivities by taking the ratios. Standard deviations for the ratios were calculated using rules for propagation of error. The adsorption isotherms of CH₄, N₂, and H₂ at 273 K, 1 bar for both C-PTHQ-800 and EC-PTHQ-1000 are shown in FIG. 19A-B. The maximum gas uptake values are summarized in Table 6.

TABLE 6
Maximum gas adsorption data for
CO₂, H₂, N₂ and CH₄ at 273 K / 1 bar.

| | CO₂ | H₂ | CH₄ | N₂ |
|---|---|---|---|---|
| | [mmol | [mmol | [mmol | [mmol |
| sample | g⁻¹] | g⁻¹] | g⁻¹] | g⁻¹] |
| C-PTHQ-800 | 4.41 | 0.08 | 0.82 | 0.37 |
| EC-PTHQ-1000 | 11.20 | 0.06 | 1.72 | 0.25 |

The isotherms were fitted with a Virial-type equation that allowed calculation of the Henry's constants, K_{H}, for each gas and material. Then, selectivities were calculated as the ratios of the K_{H} values (see Table 7), and are compared in bar-graph forms in FIG. 19C.

TABLE 7
Henry's constants and relative selectivity data for CO₂, H₂, N₂ and CH₄ at 273 K/1 bar.

| | Henry's constants [mmol g⁻¹ mbar⁻¹] ^{a, b} | | | | Relative selectivities ^{a, c} | | | |
|---|---|---|---|---|---|---|---|---|
| sample | K_{H}_{_{—}}_{CO2} | K_{H}_{_{—}}_{H2} | K_{H}_{_{—}}_{N2} | K_{H}_{_{—}}_{CH4} | CO₂/H₂ | CO₂/N₂ | CO₂/CH₄ | CH₄/H₂ |
| C-PTHQ-800 | 53 ± 8 | 0.13 ± 0.01 | 0.78 ± 0.09 | 0.68 ± 0.15 | 410 ± 75 | 68 ± 13 | 78 ± 21 | 5 ± 1 |
| EC-PTHQ-1000 | 20 ± 4 | 0.04 ± 0.007 | 0.37 ± 0.10 | 3.1 ± 0.59 | 407 ± 104 | 52 ± 18 | 6 ± 2 | 64 ± 16 |

^{a} Average of at least three measurements.
^{b} Henry's constants (K_{H}) were obtained by applying Virial-type fitting to the isotherms at 273 K (see Experimental Section in the main article).
^{c} Adsorption selectivities were calculated by taking the ratios of the corresponding K_{H} values. Errors in selectivities were calculated by applying propagation of error rules to the ratios of the K_{H} values.

1. A compound comprising moiety (I): 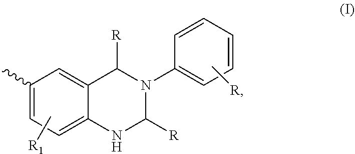
where: [symbol]
represents the point of attachment of the moiety to the rest of the compound;
each R is individually chosen from hydrogen and alkyls; and
each R₁ is individually chosen from hydrogen, halogens, and alkyls.

2. The compound of claim 1, said compound comprising the structure

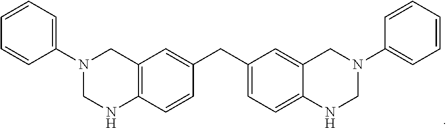 [SMILES: c1ccc(N2CNc3ccc(Cc4ccc5c(c4)CN(c4ccccc4)CN5)cc3C2)cc1]

3. A polymer derived from the compound of claim 1.

4. A polymer comprising recurring units chosen from one or more of

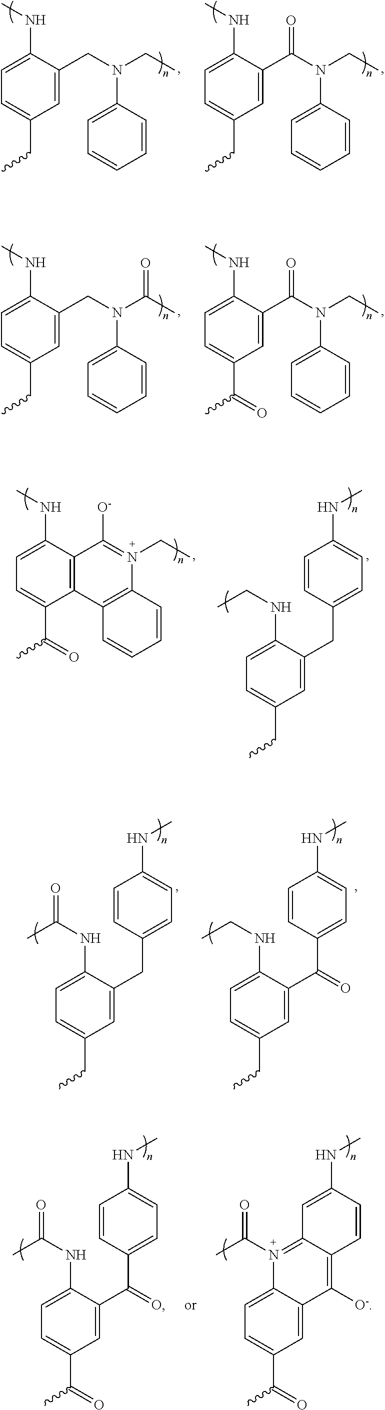

5. A gel comprising the polymer of claim 3.

6. The gel of claim 5, wherein the gel is an aerogel.

7. The gel of claim 6, said polymer comprising recurring units chosen from one or more of

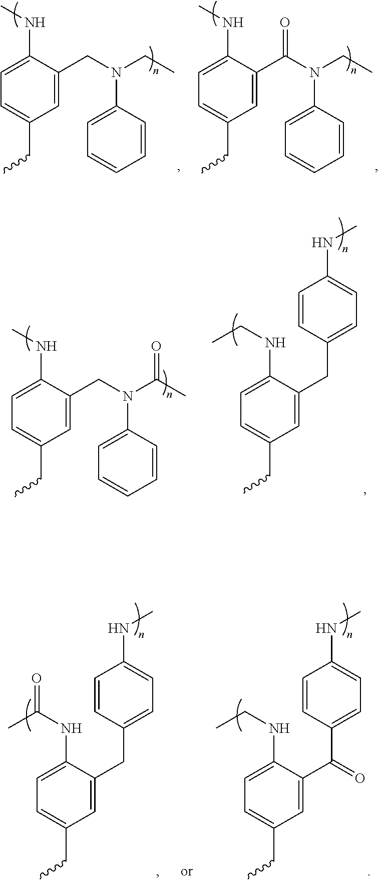

8. The gel of claim 7, said aerogel having a Brunauer-Emmett-Teller (BET) surface area of about 5 m²/g to about 30 m²/g.

9. The gel of claim 7, said aerogel having an average micropore surface area of about 1 m²/g to about 5m²/g.

10. The gel of claim 6, said polymer comprising recurring units chosen from one or more of

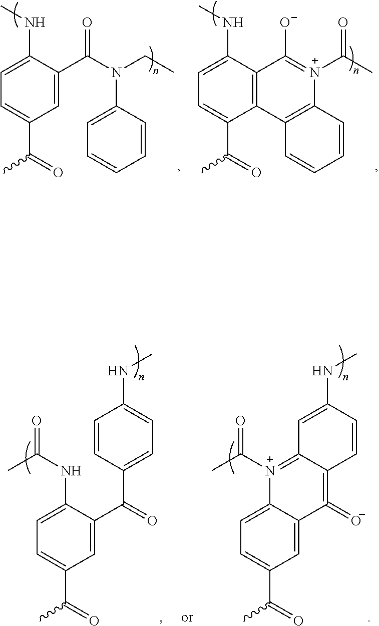

11. The gel of claim 10, said aerogel having a BET surface area of about 30 m²/g to about 60 m²/g.

12. The gel of claim 10, said aerogel having an average micropore surface area of about 3 m²/g to about 6 m²/g.

13. An aerogel derived from the aerogel of claim 10, said aerogel comprising at least about 83.5% by weight of carbon, based upon the total weight of the aerogel taken as 100% by weight.

14. The aerogel of claim 13, said aerogel having a BET surface area of about 200 m²/g to about 400 m²/g.

15. The aerogel of claim 13, said aerogel having an average micropore surface area of about 200 m²/g to about 300 m²/g.

16. The aerogel of claim 13, said aerogel having a CO₂ sorption capacity of about 2 mmol/g to about 8 mmol/g.

17. An aerogel derived from the aerogel of claim 10, said aerogel comprising at least about 84.5% by weight of carbon, based upon the total weight of the aerogel taken as 100% by weight.

18. The aerogel of claim 17, said aerogel having a BET surface area of about 1,500 m²/g to about 1,800 m²/g.

19. The aerogel of claim 17, said aerogel having an average micropore surface area of about 500 m²/g to about 900 m²/g.

20. The aerogel of claim 17, said aerogel having a CO₂ sorption capacity of about 6 mmol/g to about 16 mmol/g.

21. (canceled)

22. A process for producing a gel, said process comprising subjecting a compound according to claim 1 to ring-opening polymerization, wherein said subjecting forms said gel.

23-25. (canceled)

26. The process of claim 22, further comprising drying said gel with a supercritical fluid.

27. (canceled)

28. The process of claim 26, wherein said drying said gel with a supercritical fluid forms a partially oxidized polymer aerogel.

29. The process of claim 28, further comprising subjecting said partially oxidized polymer aerogel to heat treatment in air or under oxygen.

30-31. (canceled)

32. The process of claim 28, wherein said subjecting forms a fully oxidized polymer aerogel.

33. The process of claim 32, further comprising heating said fully oxidized polymer aerogel to a temperature of from about 700° C. to about 950° C.

34-44. (canceled)
